SEQUENCE LISTING (10 sequences)

SEQ ID NO: 1 - DNA, 904 bp, Meloidogyne incognita PPPT
  CDS (61)...(759)
gcctgcaggtcgacactagtggatccaaaggtttgagcttcatctttaactggaatagccatgtttttcaatcgtgcagctactgctccttttaaagaccggcatgatgccgggcaaaaattggctgaagctttaaagaattttaaatctcaaagggacaaagttgtggtcctagcattgccgagaggaggtgtgcctgtggcttttgaagtggcaaaatcgttgggggcacctttggatttattaatggttcgcaaaatcggtgctccaggacatgaagaatatggaataggtgctgtagttgaaggtaaccctccagaattggttatgaatgaagatgctgttaaatacactcaacccccagagggatatgttcaagcaatgatggaaaaacaattaaaggaaattgcacgccaaagaaatcgttatttgggtgaccgacctcctctttcacttgccggcaaaatcgcaattgttgttgatgatggaatagctacgggagggacagcaagagtagcaatgaaagcattacgtcaaaagaatgttgcaaaggcattgctggcatcccctttagctccttctgatactcttgccgaacttcgtgcagaaggcaatgaagtacttgttcttgaaacccctccaaatttttctgctgtcggacttcattatacaaaatttgaccagactagtgatgaggaagtaattgattgcttggaaaaatcgagggaatggttgccaaagaataatgatttaaagaattaagtttgtaattttcacattttattttgattttatttaaattgctactatttttgtagttgtaaaaaccaaaaaatattttgcaattattttacagattatttaaataataaaaaattaataattaaaaaaaaaaaaaaaaaaa

SEQ ID NO: 2 - DNA, 874 bp, Meloidogyne javanica PPPT
  CDS (25)...(711)
  misc_feature (1)...(874): n = A,T,C or G
ggtttaattacccaagtttgagcaatgtttttgggagctcgttcagctcttttcattgatcgtaaagatgccggccaaaaattggctaaggctttagcccatattttgcctcaacgtgataacattgtggttttggcacttccgcgtggcggagttccagtggcttgtgaagccgctgatgcctttcaggctcctctcgatcttctgatggtaagaaagataggtgctcctggtcatgaagaatatggaattggtgctgttgtggaaggaaatccgcccgagctcgttatgaatgaggaggctgtcagaattacacgaccatctgaagcatatgtgcaacagcaaatgcagaagcaactcaaagaaatggagagacagcgaaaaacatatttgggcgacaaaccgccggtgtcgctggaagggcgaattgccattgtcgtggacgacggaattgcaacgggtggcactgctcgagttgcgctcaaagctttgcgtcagaaaaatgttagtcgtgcaattttggcctctccgatggcgccttccgacactttggccgaattacgcgctgaaggaaatgaagtgctctgtttggagacaccgccgaatttcagtgcagttggactccattaccaacgcttcgatcaaaccagcgacgaagaggtcatccgctgcatggaaaaagccaaaaattggagcgaaagtcggaagaactagcaaagtgtattttacacggtcaatttttgtntgacccaagccccacgagagtatttatcatgtgatcacatccttctctttgagaatcacatttaaattgtgccatattcggcattacaaacaataattaatgaagttaacaaaaaaaaaaaaaaaaaaa

SEQ ID NO: 3 - DNA, 874 bp, Heterodera glycines PPPT; identical to SEQ ID NO: 2
  CDS (25)...(711)
  misc_feature (1)...(874): n = A,T,C or G

SEQ ID NO: 4 - protein, 233 aa, Meloidogyne incognita PPPT
MFFNRAATAPFKDRHDAGQKLAEALKNFKSQRDKVVVLALPRGGVPVAFEVAKSLGAPLDLLMVRKIGAPGHEEYGIGAVVEGNPPELVMNEDAVKYTQPPEGYVQAMMEKQLKEIARQRNRYLGDRPPLSLAGKIAIVVDDGIATGGTARVAMKALRQKNVAKALLASPLAPSDTLAELRAEGNEVLVLETPPNFSAVGLHYTKFDQTSDEEVIDCLEKSREWLPKNNDLKN

SEQ ID NO: 5 - protein, 229 aa, Meloidogyne javanica PPPT
MFLGARSALFIDRKDAGQKLAKALAHILPQRDNIVVLALPRGGVPVACEAADAFQAPLDLLMVRKIGAPGHEEYGIGAVVEGNPPELVMNEEAVRITRPSEAYVQQQMQKQLKEMERQRKTYLGDKPPVSLEGRIAIVVDDGIATGGTARVALKALRQKNVSRAILASPMAPSDTLAELRAEGNEVLCLETPPNFSAVGLHYQRFDQTSDEEVIRCMEKAKNWSESRKN

SEQ ID NO: 6 - protein, 229 aa, Heterodera glycines PPPT; identical to SEQ ID NO: 5

SEQ ID NO: 7 - DNA, 702 bp, Meloidogyne incognita PPPT
atgtttttcaatcgtgcagctactgctccttttaaagaccggcatgatgccgggcaaaaattggctgaagctttaaagaattttaaatctcaaagggacaaagttgtggtcctagcattgccgagaggaggtgtgcctgtggcttttgaagtggcaaaatcgttgggggcacctttggatttattaatggttcgcaaaatcggtgctccaggacatgaagaatatggaataggtgctgtagttgaaggtaaccctccagaattggttatgaatgaagatgctgttaaatacactcaacccccagagggatatgttcaagcaatgatggaaaaacaattaaaggaaattgcacgccaaagaaatcgttatttgggtgaccgacctcctctttcacttgccggcaaaatcgcaattgttgttgatgatggaatagctacgggagggacagcaagagtagcaatgaaagcattacgtcaaaagaatgttgcaaaggcattgctggcatcccctttagctccttctgatactcttgccgaacttcgtgcagaaggcaatgaagtacttgttcttgaaacccctccaaatttttctgctgtcggacttcattatacaaaatttgaccagactagtgatgaggaagtaattgattgcttggaaaaatcgagggaatggttgccaaagaataatgatttaaagaattaa

SEQ ID NO: 8 - DNA, 690 bp, Meloidogyne javanica PPPT
atgtttttgggagctcgttcagctcttttcattgatcgtaaagatgccggccaaaaattggctaaggctttagcccatattttgcctcaacgtgataacattgtggttttggcacttccgcgtggcggagttccagtggcttgtgaagccgctgatgcctttcaggctcctctcgatcttctgatggtaagaaagataggtgctcctggtcatgaagaatatggaattggtgctgttgtggaaggaaatccgcccgagctcgttatgaatgaggaggctgtcagaattacacgaccatctgaagcatatgtgcaacagcaaatgcagaagcaactcaaagaaatggagagacagcgaaaaacatatttgggcgacaaaccgccggtgtcgctggaagggcgaattgccattgtcgtggacgacggaattgcaacgggtggcactgctcgagttgcgctcaaagctttgcgtcagaaaaatgttagtcgtgcaattttggcctctccgatggcgccttccgacactttggccgaattacgcgctgaaggaaatgaagtgctctgtttggagacaccgccgaatttcagtgcagttggactccattaccaacgcttcgatcaaaccagcgacgaagaggtcatccgctgcatggaaaaagccaaaaattggagcgaaagtcggaagaactag

SEQ ID NO: 9 - DNA, 690 bp, Heterodera glycines PPPT; identical to SEQ ID NO: 8

SEQ ID NO: 10 - protein, 443 aa, Mycobacterium tuberculosis PPPT
MKLFDDRGDAGRQLAQRLAQLSGKAVVVLGLPRGGVPVAFEVAKSLQAPLDVLVVRKLGVPFQPELAFGAIGEDGVRVLNDDVVRGTHLDAAAMDAVERKQLIELQRRAERFRRGRDRIPLTGRIAVIVDDGIATGATAKAACQVARAHGADKVVLAVPIGPDDIVARFAGYADEVVCLATPALFFAVGQGYRNFTQTSDDEVVAFLDRAHRDFAEAGAIDAAADPPLRDEEVQVVAGPVPVAGHLTVPEKPRGIVVFAHGSGSSRHSIRNRYVAEVLTGAGFATLLFDLLTPEEERNRANVFDIELLASRLIDVTGWLATQPDTASLPVGYFGASTGAGAALVAAADPRVNVRAVVSRGGRPDLAGDSLGSVVAPTLLIVGGRDQVVLELNQRAQAVIPGKCQLTVVPGATHLFEEPGTLEQVAKLACDWFIDHLCGPGPSG

(19) United States
(12) Patent Application Publication
(10) Pub. No.: US 2005/0172350 A1
(43) Pub. Date: Aug. 4, 2005

(54) Nematode PPPT-like sequences

(75) Inventors: Andrew P. Kloek, St. Louis, MO (US); Deryck Jeremy Williams, St. Louis, MO (US); Brandy Salmon, Durham, NC (US)

Correspondence Address: FISH & RICHARDSON PC, 225 FRANKLIN ST, BOSTON, MA, 02110, US

(73) Assignee: Divergence, Inc. a Delaware corporation

(21) Appl. No.: 11/045,942

(22) Filed: Jan. 27, 2005

Related U.S. Application Data
(63) Continuation of application No. 10/347,776, filed on Jan. 21, 2003, now abandoned.
Application No. 10/347,776 is a division of application No. 10/113,201, filed on Mar. 29, 2002.
(60) Provisional application No. 60/280,192, filed on Mar. 30, 2001.

(51) Int. Cl. C12Q 1/68; A01K 67/027; C07H 21/04; C12N 9/16; A01H 1/00

(52) U.S. Cl. 800/18; 435/6; 435/196; 435/320.1; 435/354; 530/388.26; 536/23.2; 800/288

(57) ABSTRACT

Disclosed are a nucleic acid molecule from nematodes encoding for purine/pyrimidine phosphoribosyl transferase (PPPT) polypeptides. The PPPT-like polypeptide sequence is also provided, as are vectors, host cells, and recombinant methods for production of PPPT-like nucleotides and polypeptides. The invention further relates to screening methods for identifying inhibitors and/or activators, as well as methods for antibody production.

### RELATED APPLICATION INFORMATION

This application claims priority from provisional application Ser. No. 60/280,192, filed Mar. 30, 2001.

### BACKGROUND

Nematodes (derived from the Greek word for thread) are active, flexible, elongate, organisms that live on moist surfaces or in liquid environments, including films of water within soil and moist tissues within other organisms. While only 20,000 species of nematode have been identified, it is estimated that 40,000. to 10 million actually exist. Some species of nematodes have evolved as very successful parasites of both plants and animals and are responsible for significant economic losses in agriculture and livestock and for morbidity and mortality in humans (Whitehead (1998) Plant Nematode Control CAB International, New York).

Nematode parasites of plants can inhabit all parts of plants, including roots, developing flower buds, leaves, and stems. Plant parasites are classified on the basis of their feeding habits into the broad categories: migratory ectoparasites, migratory endoparasites, and sedentary endoparasites. Sedentary endoparasites, which include the root knot nematodes (Meloidogyne) and cyst nematodes (Globodera and Heterodera) induce feeding sites and establish long-term infections within roots that are often very damaging to crops (Whitehead, supra). It is estimated that parasitic nematodes cost the horticulture and agriculture industries in excess of $78 billion worldwide a year, based on an estimated average 12% annual loss spread across all major crops. For example, it is estimated that nematodes cause soybean losses of approximately $3.2 billion annually worldwide (Barker et al. (1994) Plant and Soil Nematodes: Societal Impact and Focus for the Future: The Committee on National Needs and Priorities in Nematology Cooperative State Research Service, US Department of Agriculture and Society of Nematologists). Several factors make the need for safe and effective nematode controls urgent. Continuing population growth, famines, and environmental degradation have heightened concern for the sustainability of agriculture, and new government regulations may prevent or severely restrict the use of many available agricultural anthelmintic agents.

The situation is particularly dire for high value crops such as strawberries and tomatoes where chemicals have been used extensively to control soil pests. The soil fumigant methyl bromide has been used effectively to reduce nematode infestations in a variety of these specialty crops. It is however regulated under the U.N. Montreal Protocol as an ozone-depleting substance and is scheduled for elimination in 2005 in the US (Carter (2001) California Agriculture 55(3):2). It is expected that strawberry and other commodity crop industries will be significantly impacted if a suitable replacement for methyl bromide is not found. Presently there are a very small array of chemicals available to control nematodes and they are frequently inadequate, unsuitable, or too costly for some crops or soils (Becker (1999) Agricultural Research Magazine 47(3):22-24; U.S. Pat. No. 6,048,714). The few available broad-spectrum nematicides such as Telone (a mixture of 1,3-dichloropropene and chloropicrin) have significant restrictions on their use because of toxicological concerns (Carter (2001) California Agriculture 55(3):12-18).

Fatty acids are a class of natural compounds that have been investigated as alternatives to the toxic, non-specific organophosphate, carbamate and fumigant pesticides (Stadler et al. (1994) Planta Medica 60(2):128-132; U.S. Pat. Nos. 5,192,546; 5,346,698; 5,674,897; 5,698,592; and 6,124,359). It has been suggested that fatty acids derive their pesticidal effects by adversely interfering with the nematode cuticle or hypodermis via a detergent (solubilization) effect, or through direct interaction of the fatty acids and the lipophilic regions of target plasma membranes (Davis et al. (1997) Journal of Nematology 29(4S):677-684). In view of this general mode of action it is not surprising that fatty acids are used in a variety of pesticidal applications including as herbicides (e.g., SCYTHE by Dow Agrosciences is the C9 saturated fatty acid pelargonic acid), as bactericides and fungicides (U.S. Pat. Nos. 4,771,571 and 5,246,716) and as insecticides (e.g., SAFER INSECTICIDAL SOAP by Safer, Inc.).

The phytotoxicity of fatty acids has been a major constraint on their general use in agricultural applications (U.S. Pat. No. 5,093,124) and the mitigation of these undesirable effects while preserving pesticidal activity is a major area of research. The esterification of fatty acids can significantly decrease their phytotoxicity (U.S. Pat. Nos. 5,674,897; 5,698,592; and 6,124,359). Such modifications can however lead to dramatic loss of nematicidal activity as is seen for linoleic, linolenic and oleic acid (Stadler et al. (1994) Planta Medica 60(2):128-132) and it may be impossible to completely decouple the phytotoxicity and nematicidal activity of pesticidal fatty acids because of their non-specific mode of action. Perhaps not surprisingly, the nematicidal fatty acid pelargonic acid methyl ester (U.S. Pat. Nos. 5,674,897; 5,698,592; and 6,124,359) shows a relatively small “therapeutic window” between the onset of pesticidal activity and the observation of significant phytotoxicity (Davis et al. (1997) J Nenatol 29(4S):677-684). This is the expected result if both the phytotoxicity and the nematicidial activity derive from the non-specific disruption of plasma membrane integrity. Similarly the rapid onset of pesticidal activity seen with many nematicidal fatty acids at therapeutic concentrations (U.S. Pat. Nos. 5,674,897; 5,698,592; and 6,124,359) suggests a non-specific mechanism of action, possibly related to the disruption of membranes, action potentials and neuronal activity.

Ricinoleic acid, the major component of castor oil, provides another example of the unexpected effects esterification can have on fatty acid activity. Ricinoleic acid has been shown to have an inhibitory effect on water and electrolyte absorption using everted hamster jejunal and ileal segments (Gaginella et al. (1975) J Pharmacol Exp Ther 195(2):355-61) and to be cytotoxic to isolated intestinal epithelial cells (Gaginella et al. (1977) J Pharmacol Exp Ther 201(1):259-66). These features are likely the source of the laxative properties of castor oil which is given as a purgative in humans and livestock (e.g., as a component of some deworming protocols). In contrast, the methyl ester of ricinoleic acid is ineffective at suppressing water absorption in the hamster model (Gaginella et al. (1975) J Pharmacol Exp Ther 195(2):355-61).

The macrocyclic lactones (e.g., avermectins and milbemycins) and delta-toxins from Bacillus thuringiensis (Bt) are chemicals that in principle provide excellent specificity and efficacy and should allow environmentally safe control of plant parasitic nematodes. Unfortunately, in practice, these two approaches have proven less effective for agricultural applications against root pathogens. Although certain avermectins show exquisite activity against plant parasitic nematodes these chemicals are hampered by poor bioavailability due to their light sensitivity, degradation by soil microorganisms and tight binding to soil particles (Lasota & Dybas (1990) Acta Leiden 59(1-2):217-225; Wright & Perry, Musculature and Neurobiology. In: The Physiology and Biochemistry of Free-Living and Plant-parasitic Nematodes, Perry & Wright, eds., CAB International 1998). Consequently despite years of research and extensive use against animal parasitic nematodes, mites and insects (plant and animal applications), macrocyclic lactones (e.g., avermectins and milbemycins) have never been commercially developed to control plant parasitic nematodes in the soil.

Bt delta toxins must be ingested to affect their target organ the brush border of midgut epithelial cells (Marroquin et al. (2000) Genetics 155(4):1693-1699). Consequently they are not anticipated to be effective against the dispersal, non-feeding, juvenile stages of plant parasitic nematodes in the field. These juvenile stages only commence feeding when a susceptible host has been infected, thus to be effective nematicides may need to penetrate the cuticle. In addition, soil mobility of a relatively large 65-130 kDa protein—the size of typical Bt delta toxins—is expected to be poor and delivery in planta is likely to be constrained by the exclusion of large particles by the feeding tube of certain plant parasitic nematodes such as Heterodera (Atkinson et al. (1998) Engineering resistance to plant-parasitic nematodes. In: The Physiology and Biochemistry of Free-Living and Plant-parasitic Nematodes, Perry & Wright, eds., CAB International 1998).

Many plant species are known to be highly resistant to nematodes. The best documented of these include marigolds (Tagetes spp.), rattlebox (Crotalaria spectabilis), chrysanthemums (Chrysanthenum spp.), castor bean (Ricinus communis), margosa (Azardiracta indica), and many members of the family Asteraceae (family Compositae) (Hackney & Dickerson (1975) J Nematol 7(1):84-90). The active principle(s) for this nematicidal activity has not been discovered in all of these examples. In the case of the Asteraceae, the photodynamic compound alpha-terthienyl has been shown to account for the strong nematicidal activity of the roots. Castor beans are plowed under as a green manure before a seed crop is set. However, a significant drawback of the castor plant is that the seed contains toxic compounds (such as ricin) that can kill humans, pets, and livestock and is also highly allergenic.

There remains an urgent need to develop environmentally safe, target-specific ways of controlling plant parasitic nematodes. In the specialty crop markets, economic hardship resulting from nematode infestation is highest in strawberries, bananas, and other high value vegetables and fruits. In the high-acreage crop markets, nematode damage is greatest in soybeans and cotton. There are however, dozens of additional crops that suffer from nematode infestation including potato, pepper, onion, citrus, coffee, sugarcane, greenhouse ornamentals and golf course turf grasses.

Nematode parasites of vertebrates (e.g., humans, livestock and companion animals) include gut roundworms, hookworms, pinworms, whipworms, and filarial worms. They can be transmitted in a variety of ways, including by water contamination, skin penetration, biting insects, or by ingestion of contaminated food.

In domesticated animals, nematode control or “de-worming” is essential to the economic viability of livestock producers and is a necessary part of veterinary care of companion animals. Parasitic nematodes cause mortality in animals (e.g., heartworm in dogs and cats) and morbidity as a result of the parasites' inhibiting the ability of the infected animal to absorb nutrients. The parasite-induced nutrient deficiency results in diseased livestock and companion animals (i.e., pets), as well as in stunted growth. For instance, in cattle and dairy herds, a single untreated infection with the brown stomach worm can permanently stunt an animal's ability to effectively convert feed into muscle mass or milk.

Two factors contribute to the need for novel anthelmintics and vaccines for control of parasitic nematodes of animals. First, some of the more prevalent species of parasitic nematodes of livestock are building resistance to the anthelmintic drugs available currently, meaning that these products will eventually lose their efficacy. These developments are not surprising because few effective anthelmintic drugs are available and most have been used continuously. Presently a number of parasitic species has developed resistance to most of the anthelmintics (Geents et al. (1997) Parasitology Today 13:149-151; Prichard (1994) Veterinary Parasitology 54:259-268). The fact that many of the anthelmintic drugs have similar modes of action complicates matters, as the loss of sensitivity of the parasite to one drug is often accompanied by side resistance, that is, resistance to other drugs in the same class (Sangster & Gill (1999) Parasitology Today 15(4):141-1.46). Secondly, there are some issues with toxicity for the major compounds currently available.

Human infections by nematodes result in significant mortality and morbidity, especially in tropical regions of Africa, Asia, and the Americas. The World Health Organization estimates 2.9 billion people are infected with parasitic nematodes. While mortality is rare in proportion to-total infections (180,000 deaths annually), morbidity is tremendous and rivals tuberculosis and malaria in disability adjusted life year measurements. Examples of human parasitic nematodes include hookworm, filarial worms, and pinworms. Hookworm is the major cause of anemia in millions of children, resulting in growth retardation and impaired cognitive development. Filarial worm species invade the lymphatics, resulting in permanently swollen and deformed limbs (elephantiasis) and invade the eyes causing African Riverblindness. Ascaris lumbricoides, the large gut roundworm infects more than one billion people worldwide and causes malnutrition and obstructive bowl disease. In developed countries, pinworms are common and often transmitted through children in daycare.

Even in asymptomatic parasitic infections, nematodes can still deprive the host of valuable nutrients and increase the ability of other organisms to establish secondary infections. In some cases, infections can cause debilitating illnesses and can result in anemia, diarrhea, dehydration, loss of appetite, or death.

While public health measures have nearly eliminated one tropical nematode (the water-borne Guinea worm), cases of other worm infections have actually increased in recent decades. In these cases, drug intervention provided through foreign donations or purchased by those who can afford it remains the major means of control. Because of the high rates of reinfection after drug therapy, vaccines remain the best hope for worm control in humans. There are currently no vaccines available.

Until safe and effective vaccines are discovered to prevent parasitic nematode infections, anthelmintic drugs will continue to be used to control and treat nematode parasitic infections in both humans and domestic animals. Finding effective compounds against parasitic nematodes has been complicated by the fact that the parasites have not been amenable to culturing in the laboratory. Parasitic nematodes are often obligate parasites (i.e., they can only survive in their respective hosts, such as in plants, animals, and/or humans) with slow generation times. Thus, they are difficult to grow under artificial conditions, making genetic and molecular experimentation difficult or impossible. To circumvent these limitations, scientists have used Caenorhabidits elegans as a model system for parasitic nematode discovery efforts. C. elegans is a small free-living bacteriovorous nematode that for many years has served as an important model system for multicellular animals (Burglin (1998) Int. J. Parasitol., 28(3): 395-41 1). The genome of C. elegans has been completely sequenced and the nematode shares many general developmental and basic cellular processes with vertebrates (Ruvkin et al. (1998) Science 282: 2033-41). This, together with its short generation time and ease of culturing, has made it a model system of choice for higher eukaryotes (Aboobaker et al. (2000) Ann. Med. 32: 23-30).

Although C. elegans serves as a good model system for vertebrates, it is an even better model for study of parasitic nematodes, as C. elegans and other nematodes share unique biological processes not found in vertebrates. For example, unlike vertebrates, nematodes produce and use chitin, have gap junctions comprised of innexin rather than connexin and contain glutamate-gated chloride channels rather than glycine-gated chloride channels (Bargmann (1998) Science 282: 2028-33). The latter property is of particular relevance given that the avermectin class of drugs is thought to act at glutamate-gated chloride receptors and is highly selective for invertebrates (Martin (1997) Vet. J. 154:11-34).

A subset of the genes involved in nematode specific processes will be conserved in nematodes and absent or significantly diverged from homologues in other phyla. In other words, it is expected that at least some of the genes associated with functions unique to nematodes will have restricted phylogenetic distributions. The completion of the C. elegans genome project and the growing database of expressed sequence tags (ESTs) from numerous nematodes facilitate identification of these “nematode specific” genes. In addition, conserved genes involved in nematode-specific processes are expected to retain the same or very similar functions in different nematodes. This functional equivalence has been demonstrated in some cases by transforming C. elegans with homologous genes from other nematodes (Kwa et al. (1995) J. Mol. Biol. 246:500-10; Redmond et al. (2001) Mol. Biochem. Parasitol. 112:125-131). This sort of data transfer has been shown in cross phyla comparisons for conserved genes and is expected to be more robust among species within a phylum. Consequently, C. elegans and other free-living nematode species are likely excellent surrogates for parasitic nematodes with respect to conserved nematode processes.

Many expressed genes in C. elegans and certain genes in other free-living nematodes can be genetically “knocked out” using RNA interference (RNAi), a technique that provides a powerful experimental tool for the study of gene function in nematodes (Fire et al. (1998) Nature 391(6669):806-811; Montgomery et al. (1998) Proc. Natl. Acad Sci USA 95(26):15502-15507). Treatment of a nematode with double-stranded RNA of a selected gene can destroy expressed sequences corresponding to the selected gene thus reducing expression of the corresponding protein. By preventing the translation of specific proteins, their functional significance and essentiality to the nematode can be assessed. Determination of essential genes and their corresponding proteins using C. elegans as a model system will assist in the rational design of anti-parasitic nematode control products.

### SUMMARY

The invention features nucleic acid molecules encoding Meloidogyne incognita, Meloidogyne javanica, and Heterodera glycines purine/pyrimidine phosphoribosyltransferase (PPPT) and other nematode PPPT-like polypeptides. M incognita and M. javanica are Root Knot Nematodes that cause substantial damage to several crops, including cotton, tobacco, pepper, and tomato. H. glycines, referred to as Soybean Cyst Nematode, is a major pest of soybean. In part, the PPPT-like nucleic acids and polypeptides of the invention allow for the identification of a nematode species, and for the identification of compounds that bind to or alter the activity of PPPT-like polypeptides. Such compounds may provide a means of combating diseases and infestations caused by nematodes, particularly by M. incognita and M. javanica (e.g., in tobacco, cotton, pepper, or tomato plants) and by H. glycines (e.g., in soybean).

The invention is based, in part, on the identification of a cDNA encoding M. incognita PPPT (SEQ ID NO: 1). This 904 nucleotide cDNA has a 699 nucleotide open reading frame (SEQ ID NO: 7) encoding a 233 amino acid polypeptide (SEQ ID NO: 4).

The invention is also based, in part, on the identification of a cDNA encoding M. javanica PPPT (SEQ ID NO: 2). This 899 nucleotide cDNA has a 699 nucleotide open reading frame (SEQ ID NO: 8) encoding a 233 amino acid polypeptide (SEQ ID NO: 5).

The invention is also based, in part, on the identification of a cDNA encoding H. glycines PPPT (SEQ ID NO: 3). This 874 nucleotide cDNA has a 687 nucleotide open reading frame (SEQ ID NO: 9) encoding a 229 amino acid polypeptide (SEQ ID NO: 6).

In one aspect, the invention features novel nematode purine/pyrimidine phosphoribosyl transferase (PPPT)-like polypeptides. Such polypeptides include purified polypeptides having the amino acid sequences set forth in SEQ ID NO: 4, 5, and/or 6. Also included are polypeptides having an amino acid sequence that is at least about 60%, 70%, 75%, 80%, 85%, 90%, 95%, or 98% identical to SEQ ID.NO: 4, 5, and/or 6. The purified polypeptides can further include a heterologous amino acid sequence, e.g., an amino-terninal or carboxy-terminal sequence. Also featured are purified polypeptide fragments of the aforementioned PPPT-like polypeptides, e.g., a fragment of at least about 20, 30, 40, 50, 75, 85, 100, 125, 140, 150, 165, 200, 229, 233 amino acids and polypeptides comprising, consisting of, or consisting essentially of such fragments. Non-limiting examples of such fragments include: fragments from about amino acid 1 to 85, 1 to 120, 1 to 140, 1 to 170, 61 to 180, 85 to 229, 121 to 233, 140 to 233, 165 to 233 and 171 to 229 of SEQ ID NO: 4, 5, and/or 6. Also featured are purified polypeptide subdomains and/or domains of the aforementioned PPPT-like polypeptides. Non-limiting examples of such subdomains and/or domains include: amino acids 1 to 190, 191 to 233, 191 to 229. The polypeptide or fragment thereof can be modified, e.g., processed, truncated, modified (e.g. by glycosylation, phosphorylation, acetylation, myristylation, prenylation, palmitoylation, amidation, addition of glycerophosphatidyl inositol), or any combination of the above.

Certain PPPT-like polypeptides comprise a sequence of 233, 230, 229, 225 amino acids or fewer.

In another aspect, the invention features novel isolated nucleic acid molecules encoding nematode PPPT-like polypeptides. Such isolated nucleic acid molecules include nucleic acids having the nucleotide sequence set forth in SEQ ID NO: 1, 2, and/or 3 or SEQ ID NO: 7, 8, and/or 9. Also included are isolated nucleic acid molecules having the same sequence as or encoding the same polypeptide as a nematode PPPT-like gene.

Also featured are: 1) isolated nucleic acid molecules having a strand that hybridizes under low stringency conditions to a single stranded probe of the sequences of SEQ ID NO: 1, 2, and/or 3 or their complements and, optionally, encodes polypeptides of between 225 and 229 or 233 amino acids; 2) isolated nucleic acid molecules having a strand that hybridizes under high stringency conditions to a single stranded probe of the sequence of SEQ ID NO: 1, 2, and/or 3 or their complements and, optionally, encodes polypeptides of between 225 and 229 or 233 amino acids; 3) isolated nucleic acid fragments of a PPPT-like nucleic acid molecule, e.g., a fragment of SEQ ID NO: 1, 2, and/or 3 that is about 190,435, 485, 500, 550, 600, 650, 750, 874, 899, and 904, or more nucleotides in length or ranges between such lengths; and 4) oligonucleotides that are complementary to a PPPT-like nucleic acid molecule or a PPPT-like nucleic acid complement, e.g., an oligonucleotide of about 10, 15, 18, 20, 22, 24, 28, 30, 35, 40, 50, 60, 70, 80, or more nucleotides in length. Exemplary oligonucleotides are oligonucleotides which anneal to a site located between a) nucleotides about 1 to 24, 1 to 48, 1 to 60, 1 to 120, 24 to 48,24 to 60, 49 to 60, 61 to 180, 721 to 780, 751 to 810, 781 to 840, 811 to 870, 841 to 904 of SEQ ID NO: 1,2, and/or 3. Nucleic acid fragments include the following non-limiting examples: nucleotides about 1 to 500, 250 to 750, 500 to 874, 500 to 899, and 500 to 904 of SEQ ID NO: 1, 2, and/or 3. Also within the invention are nucleic acid molecules that hybridize under stringent conditions to nucleic acid molecule comprising SEQ IN NO:1, 2 or 3 and comprise 3,000, 2,000, 1,000 or fewer nucleotides. The invention also includes nucleic acid molecules comprising, consisting of, or consisting essentially of such nucleic acid molecules. The isolated nucleic acid can further include a heterologous promoter operably linked to the PPPT-like nucleic acid molecule.

A molecule featured herein can be from a nematode of the class Araeolaimida, Ascaridida, Chromadorida, Desmodorida, Diplogasterida, Monhysterida, Mononchida, Oxyurida, Rhigonematida, Spirurida, Enoplia, Desmoscolecidae, Rhabditida, or Tylenchida.

In another aspect, the invention features a vector, e.g., a vector containing an aforementioned nucleic acid. The vector can further include one or more regulatory elements, e.g., a heterologous promoter. The regulatory elements can be operably linked to the PPPT-like nucleic acid molecules in order to express a PPPT-like nucleic acid molecule. In yet another aspect, the invention features a transgenic cell or transgenic organism having in its genome a transgene containing an aforementioned PPPT-like nucleic acid molecule and a heterologous nucleic acid, e.g., a heterologous promoter.

In still another aspect, the invention features an antibody, e.g., an antibody, fragment, or derivative thereof that binds specifically to an aforementioned polypeptide. Such antibodies can be polyclonal or monoclonal antibodies. The antibodies can be modified, e.g., humanized rearranged as a single-chain, or CDR-grafted. The antibodies may be directed against a fragment, a peptide, or a discontinuous epitope from a PPPT-like polypeptide.

In another aspect, the invention-features a method of screening for a compound that binds to a nematode PPPT-like polypeptide, e.g., an aforementioned polypeptide. The method includes providing the nematode polypeptide; contacting a test compound to the polypeptide; and detecting binding of the test compound to the nematode polypeptide. In one embodiment, the method further includes contacting the test compound to a plant or mammalian PPPT-like polypeptide; and detecting binding of the test compound to the plant or mammalian PPPT-like polypeptide. A test compound that binds the nematode PPPT-like polypeptide with at least 2-fold, 5-fold, 10-fold, 20-fold, 50-fold, or 100-fold affinity greater relative to its affinity for the plant or mammalian PPPT-like polypeptide can be identified. In another embodiment, the method further includes contacting the test compound to the nematode PPPT-like polypeptide; and detecting a PPPT-like activity. A decrease in the level of PPPT-like activity of the polypeptide relative to the level of PPPT-like activity of the polypeptide in the absence of the test compound is an indication that the test compound is an inhibitor of the PPPT-like activity. Such inhibitory compounds are potential selective agents for reducing the viability of a nematode expressing a PPPT-like polypeptide, e.g., M. incognita, M. javanica, and/or H. glycines.

Another featured method is a method of screening for a compound that alters an activity of a PPPT-like polypeptide. The method includes providing the polypeptide; contacting a test compound to the polypeptide; and detecting a PPPT-like activity, wherein a change in PPPT-like activity relative to the PPPT-like activity of the polypeptide in the absence of the test compound is an indication that the test compound alters the activity of the polypeptide. The method can further include contacting the test compound to a plant or mammalian PPPT-like polypeptide and measuring the PPPT-like activity of the plant or mammalian PPPT-like polypeptide. A test compound that alters the activity of the nematode PPPT-like polypeptide at a given concentration and that does not substantially alter the activity of the plant or mammalian PPPT-like polypeptide at the given concentration can be identified. An additional method includes screening for both binding to a PPPT-like polypeptide and for an alteration in activity of a PPPT-like polypeptide.

Yet another featured method is a method of screening for a compound that alters the viability or fitness of a transgenic cell or organism. The transgenic cell or organism has a transgene that expresses a PPPT-like polypeptide. The method includes contacting a test compound to the transgenic cell or organism; and detecting changes in the viability or fitness of the transgenic cell or organism.

Also featured is a method of screening for a compound that alters the expression of a nematode nucleic acid encoding a PPPT-like polypeptide, e.g., a nucleic acid encoding a M. incognita, M. javanica, and/or H. glycines PPPT-like polypeptide. The method includes contacting a cell, e.g., a nematode cell, with a test compound and detecting expression of a nematode nucleic acid encoding a PPPT-like polypeptide, e.g., by hybridization to a probe complementary to the nematode nucleic acid encoding an PPPT-like polypeptide. Compounds identified by the method are also within the scope of the invention.

In yet another aspect, the invention features a method of treating a disorder (e.g., an infection) caused by a nematode, e.g., M. incognita, M. javanica, and/or H. glycines, in a subject, e.g., a host plant or host animal. The method includes administering to the subject an effective amount of an inhibitor of a PPPT-like polypeptide activity or an inhibitor of expression of a PPPT-like polypeptide. Non-limiting examples of such inhibitors include: an antisense nucleic acid (or PNA) to a PPPT-like nucleic acid, an antibody to a PPPT-like polypeptide, or a small molecule identified as a PPPT-like polypeptide inhibitor by a method described herein.

A “purified polypeptide”, as used herein, refers to a polypeptide that has been separated from other proteins, lipids, and nucleic acids with which it is naturally associated. The polypeptide can constitute at least 10, 20, 50 70, 80 or 95% by dry weight of the purified preparation.

An “isolated nucleic acid” is a nucleic acid, the structure of which is not identical to that of any naturally occurring nucleic acid, or to that of any fragment of a naturally occurring genomic nucleic acid spanning more than three separate genes. The term therefore covers, for example: (a) a DNA which is part of a naturally occurring genomic DNA molecule but is not flanked by both of the nucleic acids that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of different: (i) DNA molecules, (ii) transfected cells, or (iii) cell clones, e.g., as these occur in a DNA library such as a cDNA or genomic DNA library. Isolated nucleic acid molecules according to the present invention further include molecules produced synthetically, as well as any nucleic acids that have been altered chemically and/or that have modified backbones.

Although the phrase “nucleic acid molecule” primarily refers to the physical nucleic acid molecule and the phrase “nucleic acid sequence” refers to the sequence of the nucleotides in the nucleic acid molecule, the two phrases can be used interchangeably.

The term “substantially pure” as used herein in reference to a given polypeptide means that the polypeptide is substantially free from other biological macromolecules. The substantially pure polypeptide is at least 75% (e.g., at least 80, 85, 95, or 99%) pure by dry weight. Purity can be measured by any appropriate standard method, for example, by column chromatography, polyacrylamide gel electrophoresis, or HPLC analysis.

The “percent identity” of two amino acid sequences or of two nucleic acids is determined using the algorithm of Karlin and Altschul (1990) Proc. Natl. Acad. Sci. USA 87:2264-2268, modified as in Karlin and Altschul (1993) Proc. Natl. Acad. Sci. USA 90:5873-77. Such an algorithm is incorporated into the BLASTN and BLASTX programs (version 2.0) of Altschul et al. (1990)J. Mol. Biol. 215:403-410. BLAST nucleotide searches can be performed with the BLASTN program, score=100, wordlength=-12 to obtain nucleotide sequences homologous to the nucleic acid molecules of the invention. BLAST protein searches can be performed with the BLASTX program, score=50, wordlength=3 to obtain amino acid sequences homologous to the protein molecules of the invention. Where gaps exist between two sequences, Gapped BLAST can be utilized as described in Altschul et al. (1997) Nucleic Acids Res. 25:3389-3402. When utilizing BLAST and Gapped BLAST programs, the default parameters of the respective programs (e.g., BLASTX and BLASTN) can be used (available on the Internet at ncbi.nlm.nih.gov).

As used herein, the term “transgene” means a nucleic acid sequence (encoding, e.g., one or more subject polypeptides), which is partly or entirely heterologous, i.e., foreign, to the transgenic plant, animal, or cell into which it is introduced, or, is homologous to an endogenous gene of the transgenic plant, animal, or cell into which it is introduced, but which is designed to be inserted, or is inserted, into the plant's genome in such a way as to alter the genome of the cell into which it is inserted (e.g., it is inserted at a location which differs from that of the natural gene or its insertion results in a knockout). A transgene can include one or more transcriptional regulatory sequences and other nucleic acid sequences, such as introns, that may be necessary for optimal expression of the selected nucleic acid, all operably linked to the selected nucleic acid, and may include an enhancer sequence.

As used herein, the term “transgenic cell” refers to a cell containing a transgene.

As used herein, a “transgenic plant” is any plant in which one or more, or all, of the cells of the plant includes a transgene. The transgene can be introduced into the cell, directly or indirectly by introduction into a precursor of the cell, by way of deliberate genetic manipulation, such as by T-DNA mediated transfer, electroporation, or protoplast transformation. The transgene may be integrated within a chromosome, or it may be extrachromosomally replicating DNA.

As used herein, the term “tissue-specific promoter” means a DNA sequence that serves as a promoter, i.e., regulates expression of a selected DNA sequence operably linked to the promoter, and which effects expression of the selected DNA sequence in specific cells of a tissue, such as a leaf, root, or stem.

As used herein, the terms “hybridizes under stringent conditions” and “hybridizes under high stringency conditions” refers to conditions for hybridization in 6× sodium chloride/sodium citrate (SSC) buffer at about 45° C., followed by two washes in 0.2×SSC buffer, 0.1% SDS at 60° C. or 65° C. As used herein, the term “hybridizes under low stringency conditions” refers to conditions for hybridization in 6×SSC buffer at about 45° C., followed by two washes in 6×SSC buffer, 0.1% (w/v) SDS at 50° C.

A “heterologous promoter”, when operably linked to a nucleic acid sequence, refers to a promoter which is not naturally associated with the nucleic acid sequence.

As used herein, an agent with “antihelminthic activity” is an agent, which when tested, has measurable nematode-killing activity or results in infertility or sterility in the nematodes such that unviable or no offspring result. In the assay, the agent is combined with nematodes, e.g., in a well of microtiter dish having agar media or in the soil containing the agent. Staged adult nematodes are placed on the media. The time of survival, viability of offspring, and/or the movement of the nematodes are measured. An agent with “antihelminthic activity” reduces the survival time of adult nematodes relative to unexposed similarly-staged adults, e.g., by about 20%, 40%, 60%, 80%, or more. In the alternative, an agent with “antihelminthic activity” may also cause the nematodes to cease replicating, regenerating, and/or producing viable progeny, e.g., by about 20%, 40%, 60%, 80%, or more.

As used herein, the term “binding” refers to the ability of a first compound and a second compound that are not covalently attached to physically interact. The apparent dissociation constant for a binding event can be I mM or less, for example, 10 nM, 1 nM, 0.1 nM or less.

As used herein, the term “binds specifically” refers to the ability of an antibody to discriminate between a target ligand and a non-target ligand such that the antibody binds to the target ligand and not to the non-target ligand when simultaneously exposed to both the given ligand and non-target ligand, and when the target ligand and the non-target ligand are both present in molar excess over the antibody.

As used herein, the term “altering an activity” refers to a change in level, either an increase or a decrease in the activity, particularly a PPPT-like or PPPT activity. The change can be detected in a qualitative or quantitative observation. If a quantitative observation is made, and if a comprehensive analysis is performed over a plurality of observations, one skilled in the art can apply routine statistical analysis to identify modulations where a level is changed and where the statistical parameter, the p value, is less than 0.05.

In part, the nematode PPPT proteins and nucleic acids described herein are novel targets for anti-nematode vaccines, pesticides, and drugs. Inhibition of these molecules can provide means of inhibiting nematode metabolism and/or the nematode life-cycle.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts the cDNA sequence of M. incognita PPPT (SEQ ID NO: 1), its corresponding encoded amino acid sequence (SEQ ID NO: 4), and its open reading frame (SEQ ID NO: 7).

FIG. 2 depicts the cDNA sequence of M. javanica PPPT (SEQ ID NO: 2), its corresponding encoded amino acid sequence (SEQ ID NO: 5), and its open reading frame (SEQ ID NO: 8).

FIG. 3 depicts the cDNA sequence of H. glycines PPPT (SEQ ID NO: 3), its corresponding encoded amino acid sequence (SEQ ID NO: 6), and its open reading frame (SEQ ID NO: 9).

FIG. 4 is an alignment of the sequences of M. incognita (1), M. javanica (2), and H. glycines (3) PPPT-like polypeptides (SEQ ID NO: 4, 5, and 6) and Mycobacterium tuberculosis (4) PPPT-like sequence (SEQ ID NO: 10).

### DETAILED DESCRIPTION

Pyrimidine/purine phosphoribosyl transferases (also known as PPPTs or PRTases) are enzymes involved in salvage pathways for nucleic acids and are responsible for the conversion of free pyrimidine and purine bases and nucleosides into their corresponding nucleotides. Adenine PPPTs, for example, catalyze the conversion of adenine and α-D-5-phosphoribose-1-pryrophosphate (PRPP) to adenine monophosphate (AMP) and inorganic pyrophosphate (PPi).

All protozoan parasites studied to date, as well as some parasitic trematodes, lack the ability to synthesize purine nucleotides de novo (Wang (1984) J. Med. Chem. 27:1-9). Instead, they utilize purine salvage pathways to convert the host organism's purine bases and nucleosides into the nucleotides necessary for nucleic acid metabolism. For example, purine salvage pathway enzymes have been shown to be critical for nucleic acid metabolism in Tritrichomonas foetus, an anaerobic flagellated protozoan responsible for causing urogenital trichomoniasis in cattle, and in Schistosoma mansoni, a human parasitic trematode that causes schistosomiasis, one of the most prevalent infectious diseases in the world (Wang et al. (1984) Exp. Parasitol. 57:68-75; Senft et al. (1983) Pharmacol. Ther. 20:341-356; Dovey et al. (1984) Mol. Biochem. Parasitol. 11:157-167

PPPTs are potentially promising targets for anti-parasitic therapy. While mammals can produce purine nucleotides de novo, they can also make use of purine salvage pathways. Thus, it is desirable to provide compounds that interfere with parasite PPPTs (e.g., inhibit expression or activity) without substantially interfering with the corresponding mammalian enzymes.

Several studies have made strides in identifying specific inhibitors of parasitic PPPTs. For example, the availability of crystal structures for both parasitic and human variants of the guanine PPPTs of Tritrichomonas foetus has facilitated both the rational selection and optimization of inhibitors that are both selective for the parasite enzyme in vitro and efficacious against the parasite in cell culture (Somoza et al. (1998) Biochemistry 37:5344-5348).

The putative PPPTs from Meloidogyne incognita, Meloidogyne javanica and Heterodera glycines described herein do not appear to have obvious homologs except for a class of conserved proteins in Mycobacteria and other bacterial species. Moreover, because the PPPTs of the invention do not appear to have closely related homologs in plants or vertebrates, they are targets for parasitic nematode control.

Compounds that inhibit the expression or activity of the PPPTs of the invention are potentially useful compounds for controlling parasitic nematode infection. Particularly useful compounds are those that do not significantly inhibit the expression or activity of a PPPT used by the host of the parasitic nematode.

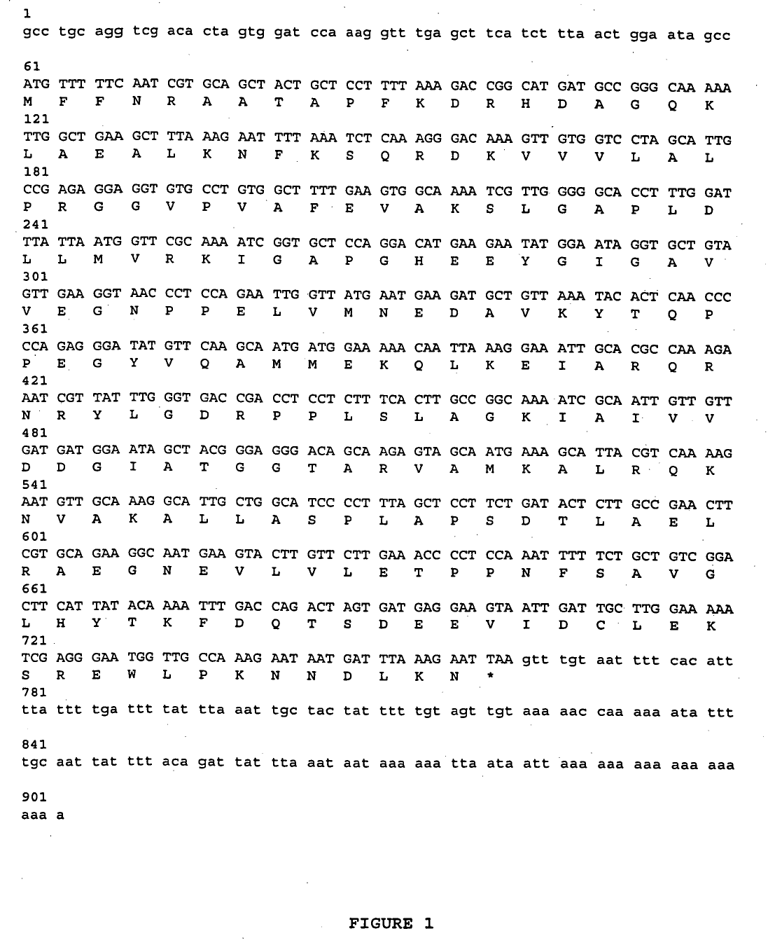

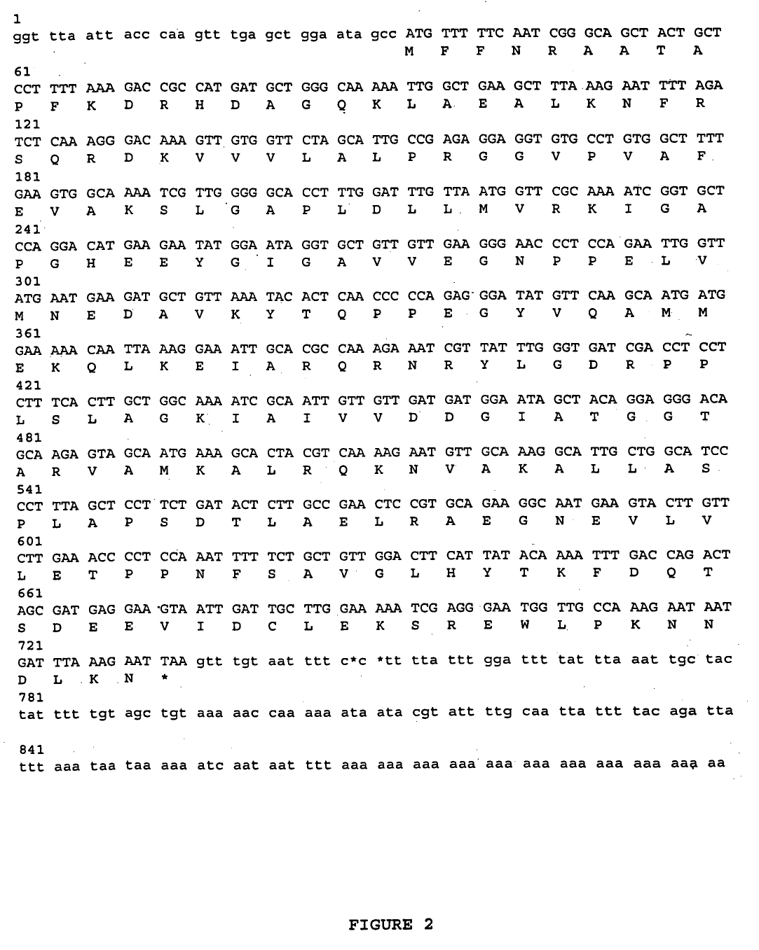

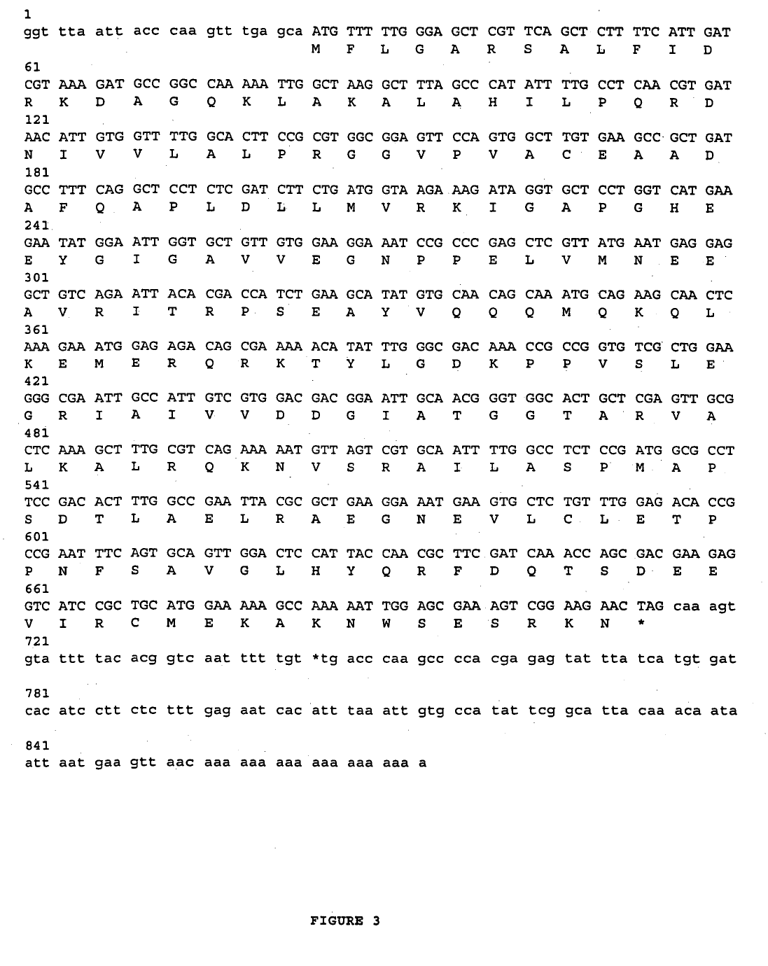

The present invention provides nucleic acids from nematodes encoding pyrimidine/purine phosphoribosyl transferases [PPPT]-like polypeptides. The M. incognita nucleic acid molecule (SEQ ID NO: 1) and the encoded pyrimidine/purine phosphoribosyl transferase [PPPT]-like polypeptide (SEQ ID NO: 4) are depicted in FIG. 1. The M. javanica nucleic acid molecule (SEQ ID NO: 2) and the encoded pyrimidine/purine phosphoribosyl transferase [PPPT]-like polypeptide (SEQ ID NO: 5) are depicted in FIG. 2. The H. glycines nucleic acid molecule (SEQ ID NO: 3) and the encoded pyrimidine/purine phosphoribosyl transferase [PPPT]-like polypeptide (SEQ ID NO: 6) are depicted in FIG. 3. Certain sequence information for the PPPT genes described herein is summarized in Table 1, below.

TABLE 1
PPPT Sequences

| Species | cDNA | ORF | Polypeptide | Fig. |
|---|---|---|---|---|
| M. incognita | SEQ ID NO: 1 | SEQ ID NO: 7 | SEQ ID NO: 4 | |
| M. javanica | SEQ ID NO: 2 | SEQ ID NO: 8 | SEQ ID NO: 5 | |
| H. glycines | SEQ ID NO: 3 | SEQ ID NO: 9 | SEQ ID NO: 6 | |

The invention is based, in part, on the discovery of this PPPT-like sequence from M. incognita, M javanica, and H. glycines. The following examples are, therefore, to be construed as merely illustrative, and not limitative of the remainder of the disclosure in any way whatsoever. All of the publications cited herein are hereby incorporated by reference in their entirety.

### EXAMPLES

Four expressed sequence tags (ESTs; short nucleic acid fragment sequences from single sequencing reads) were identified in dbest that are predicted to encode PPPT-like enzymes in three nematode species: M. incognita (GI: 7921954, 7798201, GenBank Accession No. AW783595); M. javanica (GI: 9829737; GenBank Accession No. BE578795); and H. glycines (GI: 10714612; GenBank Accession No. BF014337), all found in McCarter et al. ((1999) Washington University Nematode EST Project).

Full Length PPPT-Like cDNA Sequences

Plasmid clone Div227, corresponding to the M. incognita EST sequence (GI: 7921954) was obtained from the Genome Sequencing Center (St. Louis, Mo.). Similarly, plasmid clone Div229, corresponding to the M, javanica EST sequence (GI: 9829737), and plasmid clone Div331, corresponding to the H. glycines EST sequence (GI: 10714612), were also obtained from the Genome Sequencing Center (St. Louis, Mo.). The cDNA inserts in the plasmids were sequenced in their entirety. Unless otherwise indicated, all nucleotide sequences determined herein were sequenced with an automated DNA sequencer (such as model 373 from Applied Biosystems, Inc.) using processes well known to those skilled in the art. Primers used for sequencing are listed in Table 2, below.

The sequences of three PPPT-like nucleic acid molecules are depicted in FIG. 1, FIG. 2, and FIG. 3 as SEQ ID NO: 1, SEQ ID NO: 2, and SEQ ID NO: 3, respectively. SEQ ID NO: 1 and SEQ ID NO: 2 contain open reading frames encoding 233 amino acid polypeptides. SEQ ID NO: 3 contains an open reading frame encoding a 229 amino acid polypeptide.

TABLE 2
Primer Sequences

| | | | |
|---|---|---|---|
| Name | Sequence | SEQ ID NO: | Homology to |

| | | | |
|---|---|---|---|
| T7 | gtaatacgactcactatagggc | 11 | vector polylinker primer |
| T3 | aattaaccctcactaaaggg | 12 | vector polylinker primer |
| SL1 | gggtttaattacccaagtttga | 13 | nematode transpliced leader |
| Oligo dT | gagagagagagagagagagaactagtctcgagtttttttttttttttttt | 14 | universal primer to poly A tail |

Characterization of M. incognita. M. iavanica, and H. glycines PPPT

The sequence of the M. incognita PPPT-like cDNA (SEQ ID NO:1) is depicted in FIG. 1. This nucleotide sequence contains an open reading frame (SEQ ID NO:7) encoding a 233 amino acid polypeptide (SEQ ID NO:4). The M. incognita PPPT-like protein sequence (SEQ ID NO: 4) is approximately 44% identical to a Mycobacterium tuberculosis PPPT gene (SEQ ID NO: 10).

The sequence of the M. javanica PPPT-like cDNA (SEQ ID NO:2) is depicted in FIG. 2. This nucleotide sequence also contains an open reading frame (SEQ ID NO:8) encoding a 233 amino acid polypeptide (SEQ ID NO:5). The M. javanica PPPT-like protein sequence (SEQ ID NO: 5) is also approximately 44% identical to the Mycobacterium tuberculosis PPPT gene (SEQ ID NO: 10).

The sequence of the H. glycines PPPT-like cDNA (SEQ ID NO:3) is depicted in FIG. 3. This nucleotide sequence contains an open reading frame (SEQ ID NO:9) encoding a 229 amino acid polypeptide (SEQ ID NO:6). The H. glycines PPPT-like protein sequence (SEQ ID NO: 6) is approximately 41% identical to the M. tuberculosis PPPT gene (SEQ ID NO: 10).

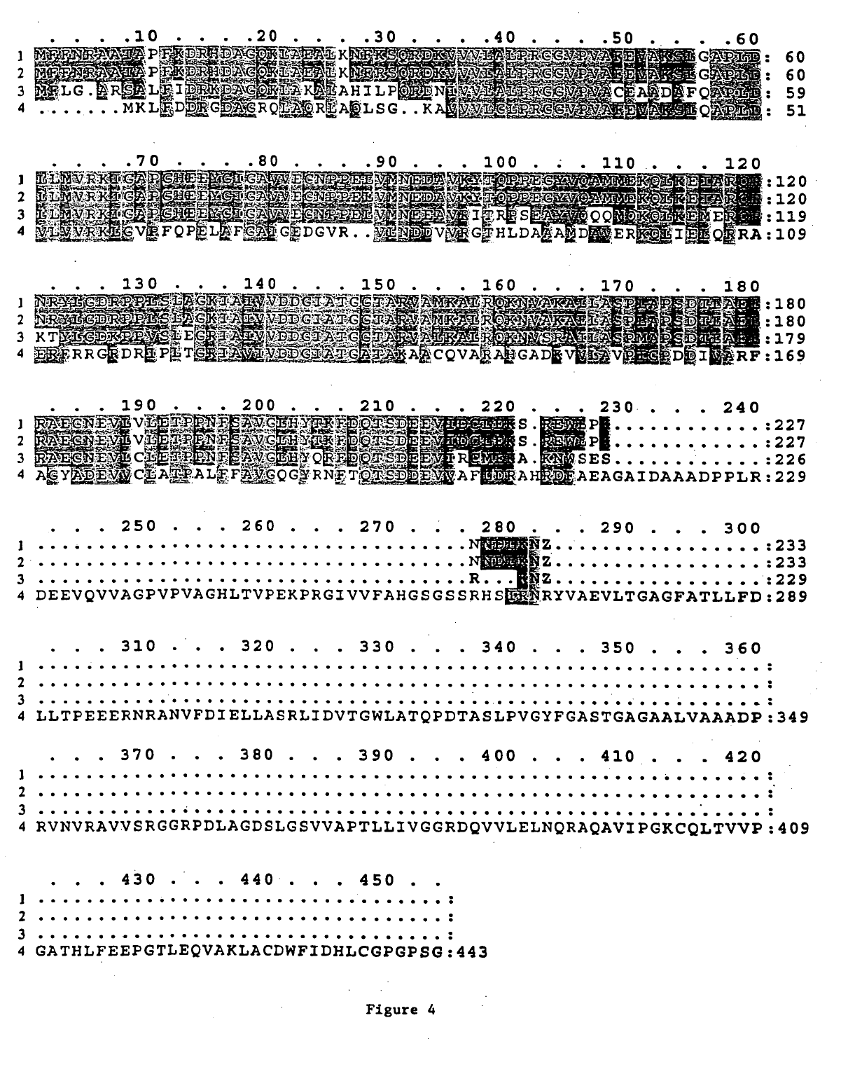

The similarity between the PPPT-like proteins from M. incognita, M. javanica, a H. glycines and M. tuberculosis is presented as a multiple alignment generated by the Clustal X multiple alignment program as described below (FIG. 4).

The similarity between M. incognita, M. javanica, and H. glycines PPPT-like sequences and other sequences was also investigated by comparison to sequence databases using BLASTP analysis against nr (a non-redundant protein sequence database (available on the Internet at ncbi.nlm.nih.gov) and TBLASTN analysis against dbest (an EST sequence database (available on the Internet at ncbi.nlm.nih.gov; top 500 hits; E=1e-4). The “Expect (E) value” is the number of sequences that are predicted to align by chance given the size of the queried database. This analysis was used to determine the potential number of plant and vertebrate homologs for each of the nematode PPPT-like polypeptides described above. M. incognita (SEQ ID NO: 1), M. javanica (SEQ ID NO: 2) and H. glycines (SEQ ID NO: 3) PPPT-like sequences had no vertebrate and/or plant hits in nr or dbest having sufficient sequence similarity to meet the threshold E value of 1e-4 (this E value approximately corresponds to a threshold for removing sequences having a sequence identity of less than about 25% over approximately 100 amino acids). Accordingly, the M. incognita, M. javanica, and H. glycines PPPT-like enzymes of this invention do not appear to share significant sequence similarity with the more common vertebrate forms of the enzyme such as the Homo sapiens pyrimidine/purine phosphoribosyl transferases GenBank® Accession No. NM 000194 (GI: 4504482) and GenBank® Accession No. AW300243 (GI: 6710009).

The PPPT-like enzymes present in M. incognita, M. javanica, and H. glycines also appear to be more closely related to PPPT enzymes present in some types of bacteria than to the PPPT enzymes present in some nematodes (e.g., C. elegans). Accordingly, the M. incognita, M. javanica, and H. glycines PPPT-like enzymes of the invention are useful targets of inhibitory compounds selective for some nematodes over their hosts (e.g., humans, animals, and plants).

Functional predictions were made with the PFAM (available on the Internet at pfam.wustl.edu),.which is a Hidden Markov Model based database of families of protein domains. Searches in pfam confirm that the nucleotide sequences in M. incognita, M. javanica, and H. glycines do encode for a pyrimidine/purine phosphoribosyl transferases. Protein localizations were predicted using the TargetP server (available on the Internet at cbs.dtu.dk/services/TargetP). The M. incognita, M. javanica, and H. glycines PPPT (SEQ ID NO: 4, 5, and 6, respectively) polypeptides are potentially cytosolic.

Identification of Additional PPPT-Like Sequences

A skilled artisan can utilize the methods provided in the example above to identify additional nematode PPPT-like sequences, e.g., PPPT-like sequence from nematodes other M. incognita, M. javanica, and/or H. glycines. In addition, nematode PPPT-like sequences can be identified by a variety of methods including computer-based database searches, hybridization-based methods, and functional complementation.

Database Identification. A nematode PPPT-like sequence can be identified from a sequence database, e.g., a protein or nucleic acid database using a sequence disclosed herein as a query. Sequence comparison programs can be used to compare and analyze the nucleotide or amino acid sequences. One such software package is the BLAST suite of programs from the National Center for Biotechnology Institute (NCBI; Altschul et al. (1997) Nuc. Acids Research 25:3389-3402.). A PPPT-like sequence of the invention can be used to query a sequence database, such as nr, dbest (expressed sequence tag (EST) sequences), and htgs (high-throughput genome sequences), using a computer-based search, e.g., FASTA, BLAST, or PSI-BLAST search. Homologous sequences in other species (e.g., humans, plants, animals, fungi) can be detected in a PSI-BLAST search of a database such as nr (E value=1e-2, H value=1e-4, using, for example, four iterations; (available on the Internet at ncbi.nlm.nih.gov). Sequences so obtained can be used to construct a multiple alignment, e.g., a ClustalX alignment, and/or to build a phylogenetic tree, e.g., in ClustalX using the Neighbor-Joining method (Saitou et al. (1987) Mol. Biol. Evol. 4:406-425) and bootstrapping (1000 replicates; Felsenstein (1985) Evolution 39:783-791). Distances may be corrected for the occurrence of multiple substitutions [D_{corr}=-1n(1-D-D²/5) where D is the fraction of amino acid differences between two sequences] (Kimura (1983) The Neutral Theory of Molecular Evolution).

The aforementioned search strategy can be used to identify PPPT-like sequences in nematodes of the following non-limiting, exemplary genera: Plant nematode genera: Afrina, Anguina, Aphelenchoides, Belonolaimus, Bursaphelenchus, Cacopaurus, Cactodera, Criconema, Criconemoides, Cryphodera, Ditylenchus, Dolichodorus, Dorylaimus, Globodera, Helicotylenchus, Henicriconemoides, Hemicycliophora, Heterodera, Hirschmanniella, Hoplolaimus, Hypsoperine, Longidorus, Meloidogyne, Mesoanguina, Nacobbus, Nacobbodera, Panagrellus, Paratrichodorus, Paratylenchus, Pratylenchus, Pterotylenchus, Punctodera, Radopholus, Rhadinaphelenchus, Rotylenchulus, Rotylenchus, Scutellonema, Subanguina, Thecavermiculatus, Trichodorus, Turbatrix, Tylenchorhynchus, Tylenchulus, Xiphinema.

Animal and human nematode genera: Acanthocheilonema, Aelurostrongylus, Ancylostoma, Angiostrongylus, Anisakis, Ascaris, Ascarops, Bunostomum, Brugia, Capillaria, Chabertia, Cooperia, Crenosoma, Cyathostome species (Small Strongyles), Dictyocaulus, Dioctophyma, Dipetalonema, Dirofiliaria, Dracunculus, Draschia, Elaneophora, Enterobius, Filaroides, Gnathostoma, Gonylonema, Habronema, Haemonchus, Hyostrongylus, Lagochilascaris, Litomosoides, Loa, Mammomonogamus, Mansonella, Muellerius, Metastrongylid, Necator, Nematodirus, Nippostrongylus, Oesophagostomum, Ollulanus, Onchocerca, Ostertagia, Oxyspirura, Oxyuris, Parafilaria, Parascaris, Parastrongyloides, Parelaphostrongylus, Physaloptera, Physocephalus, Protostrongylus, Pseudoterranova, Setaria, Spirocerca, Stephanurus, Stephanofilaria, Strongyloides, Strongylus, Spirocerca, Syngamus, Teladorsagia, Thelazia, Toxascaris, Toxocara, Trichinella, Trichostrongylus, Trichuris, Uncinaria, and Wuchereria.

Particularly preferred nematode genera include: Plant: Anguina, Aphelenchoides, Belonolaimus, Bursaphelenchus, Ditylenchus, Dolichodorus, Globodera, Heterodera, Hoplolaimus, Longidorus, Meloidogyne, Nacobbus, Pratylenchus, Radopholus, Rotylenchus, Tylenchulus, Xiphinema.

Animal and human: Ancylostoma, Ascaris, Brugia, Capillaria, Cooperia, Cyathostome species, Dictyocaulus, Dirofiliaria, Dracunculus, Enterobius, Haemonchus, Necator, Nematodirus, Oesophagostomum, Onchocerca, Ostertagia, Oxyspirura, Oxyuris, Parascaris, Strongyloides, Strongylus, Syngamus, Teladorsagia, Thelazia, Toxocara, Trichinella, Trichostrongylus, Trichuris, and Wuchereria.

Particularly preferred nematode species include: Plant: Anguina tritici, Aphelenchoides fragariae, Belonolaimus longicaudatus, Bursaphelenchus xylophilus, Ditylenchus destructor, Ditylenchus dipsaci Dolichodorus heterocephalous, Globodera pallida, Globodera rostochiensis, Globodera tabacum, Heterodera avenae, Heterodera cardiolata, Heterodera carotae, Heterodera cruciferae, Heterodera glycines, Heterodera major, Heterodera schachtii, Heterodera zeae, Hoplolaimus tylenchiformis, Longidorus sylphus, Meloidogyne acronea, Meloidogyne arenaria, Meloidogyne chitwoodi, Meloidogyne exigua, Meloidogyne graminicola, Meloidogyne hapla, Meloidogyne incognita, Meloidogyne javanica, Meloidogyne nassi, Nacobbus batatiformis, Pratylenchus brachyurus, Pratylenchus coffeae, Pratylenchus penetrans, Pratylenchus scribneri, Pratylenchus zeae, Radopholus similis, Rotylenchus reniformis, Tylenchulus semipenetrans, Xiphinema americanum.

Animal and human: Ancylostoma braziliense, Ancylostoma caninum, Ancylostoma ceylanicum, Ancylostoma duodenale, Ancylostoma tubaeforme, Ascaris suum, Ascaris lumbrichoides, Brugia malayi, Capillaria bovis, Capillaria plica, Capillariafeliscati, Cooperia oncophora, Cooperia punctata, Cyathostome species, Dictyocaulus filaria, Dictyocaulus viviparus, Dictyocaulus arnfieldi, Dirofiliaria immitis, Dracunculus insignis, Enterobius vermicularis, Haemonchus contortus, Haemonchusplacei, Necator americanus, Nematodirus helvetianus, Oesophagostomum radiatum, Onchocerca volvulus, Onchocerca cervicalis, Ostertagia ostertagi, Ostertagia circumcincta, Oxyuris equi, Parascaris equorum, Strongyloides stercoralis, Strongylus vulgaris, Strongylus edentatus, Syngamus trachea, Teladorsagia circumcincta, Toxocara cati, Trichinella spiralis, Trichostrongylus axei, Trichostrongylus colubriformis, Trichuris vulpis, Trichuris suis, Trichurs trichiura, and Wuchereria bancrofti.

Further, a PPPT-like sequence can be used to identify additional PPPT-like sequence homologs within a genome. Multiple homologous copies of a PPPT-like sequence can be present. For example, a nematode PPPT-like sequence can be used as a seed sequence in an iterative PSI-BLAST search (default parameters, substitution matrix=Blosum62, gap open=1 1, gap extend=1) of a database, such as nr or wormpep (E value=1e-2, H value=1e-4, using, for example 4 iterations) to determine the number of homologs in a database, e.g., in a database containing the complete genome of an organism. A nematode PPPT-like sequence can be present in a genome along with 1, 2, 3, 4, 5, 6, 8, 10, or more homologs.

Hybridization Methods. A nematode PPPT-like sequence can be identified by a hybridization-based method using a sequence provided herein as a probe. For example, a library of nematode genomic or cDNA clones can be hybridized under low stringency conditions with the probe nucleic acid. Stringency conditions can be modulated to reduce background signal and increase signal from potential positives. Clones so identified can be sequenced to verify that they encode PPPT-like sequences.

Another hybridization-based method utilizes an amplification reaction (e.g., the polymerase chain reaction (PCR)). Oligonucleotides, e.g., degenerate oligonucleotides, are designed to hybridize to a conserved region of a PPPT-like sequence (e.g., a region conserved in the three nematode sequences depicted in FIG. 4). The oligonucleotides are used as primers to amplify a PPPT-like sequence from template nucleic acid from a nematode, e.g., a nematode other than M. incognita, M. javanica, and/or H. glycines. The amplified fragment can be cloned and/or sequenced.

Complementation Methods. A nematode PPPT-like sequence can be identified from a complementation screen for a nucleic acid molecule that restores PPPT-like activity to a cell lacking a PPPT-like activity. Routine methods can be used to construct bacterial or yeast strains that lack specific enzymatic activities, e.g., PPPT activity. For example,. an E. coli and/or a Saccharomyces cerevisiae strain mutated at the PPPT gene locus can be identified by selecting for resistance to toxic nucleoside analogs, e.g., 8-azaadenine, 2,6-diaminopurine, and/or 2-fluoroadenine (Levine et al. (1981) Mol. Gen. Genet. 181:313-318; Sahota et al. (1987) Mutat. Res 180:81-87). Such a strain can be transformed with a plasmid library expressing nematode cDNAs. Strains are identified in which PPPT activity is restored. For example, the pppf E. coli or S. cerevisiae strains transformed with the plasmid library can be exposed to 8-azaadenine, 2,6-diaminopurine, and/or 2-fluoroadenine to select for strains that have acquired sensitivity to the analogs and are expressing a nematode PPPT-like gene. The plasmid harbored by the strain can be recovered to identify and/or characterize the inserted nematode cDNA that provides PPPT-like activity when expressed.

Full-length cDNA and Sequencing Methods. The following methods can be used, e.g., alone or in combination with another method described herein, to obtain full-length nematode PPPT-like genes and determine their sequences.

Plant parasitic nematodes are maintained on greenhouse pot cultures depending on nematode preference. Root Knot Nematodes (Meloidogyne sp) are propagated on Rutgers tomato (Burpee), while Soybean Cyst Nematodes (Heterodera sp) are propagated on soybean. Total RNA is isolated using the TRIZOL reagent (Gibco BRL). Briefly, 2 ml of packed worms are combined with 8 ml TRIZOL reagent and solubilized by vortexing. Following 5 minutes of incubation at room temperature, the samples are divided into smaller volumes and spun at 14,000×g for 10 minutes at 4□C to remove insoluble material. The liquid phase is extracted with 200 μl of chloroform, and the upper aqueous phase is removed to a fresh tube. The RNA is precipitated by the addition of 500 μl of isopropanol and centrifuged to pellet. The aqueous phase is carefully removed, and the pellet is washed in 75% ethanol and spun to re-collect the RNA pellet. The supernatant is carefully removed, and the pellet is air dried for 10 minutes. The RNA pellet is resuspended in 50 μl of DEPC-H2O and analyzed by spectrophotometry at λ260 and 280 nm to determine yield and purity. Yields can be 1-4 mg of total RNA from 2 ml of packed worms.

Full-length cDNAs can be generated using 5′ and 3′ RACE techniques in combination with EST sequence information. The molecular technique 5′ RACE (Life Technologies, Inc., Rockville, Md.) can be employed to obtain complete or near-complete 5′ ends of cDNA sequences for nematode PPPT-like cDNA sequences. Briefly, following the instructions provided by Life Technologies, first strand cDNA is synthesized from total nematode RNA using Murine Leukemia Virus Reverse Transcriptase (M-MLV RT) and a gene specific “antisense” primer, e.g., designed from available EST sequence. RNase H is used to degrade the original mRNA template. The first strand cDNA is separated from unincorporated dNTPs, primers, and proteins using a GlassMAX Spin Cartridge. Terminal deoxynucleotidyl transferase (TdT) is used to generate a homopolymeric dC tailed. extension by the sequential addition of dCTP nucleotides to the 3′ end of the first strand cDNA. Following addition of the dC homopolymeric extension, the first strand cDNA is directly amplified without further purification using Taq DNA polymerase, a gene specific “antisense” primer designed from available EST sequences to anneal to a site located within the first strand cDNA molecule, and a deoxyinosine-containing primer that anneals to the homopolymeric dC tailed region of the cDNA in a polymerase chain reaction (PCR). 5′ RACE PCR amplification products are cloned into a suitable vector for further analysis and sequencing.

The molecular technique, 3′ RACE (Life Technologies, Inc., Rockville, Md.), can be employed to obtain complete or near-complete 3′ ends of cDNA sequences for nematode PPPT-like cDNA sequences. Briefly, following the instructions provided by Life Technologies (Rockville, Md.), first strand cDNA synthesis is performed on total nematode RNA using SuperScrip™ Reverse Transcriptase and an oligo-dT primer that anneals to the polyA tail. Following degradation of the original mRNA template with RNase H, the first strand cDNA is directly PCR amplified without further purification using Taq DNA polymerase, a gene specific primer designed from available EST sequences to anneal to a site located within the first strand cDNA molecule, and a “universal” primer which contains sequence identity to 5′ end of the oligo-dT primer. The 3′ RACE PCR amplification products are cloned into a suitable vector for further analysis and sequencing.

Nucleic Acid Variants

Isolated nucleic acid molecules of the present invention include nucleic acid molecules that have an open reading frame encoding a PPPT-like polypeptide. Such nucleic acid molecules include molecules having: the sequences recited in SEQ ID NO: 1, 2, and/or 3; and sequences coding for the PPPT-like proteins recited in SEQ ID NO: 4, 5, and/or 6. These nucleic acid molecules can be used, for example, in a hybridization assay to detect the presence of a M. incognita, M. javanica, and/or H. glycines nucleic acid in a sample.

The present invention includes nucleic acid molecules such as those shown in SEQ ID NO: 1, 2, and/or 3 that may be subjected to mutagenesis to produce single or multiple nucleotide substitutions, deletions, or insertions. Nucleotide insertional derivatives of the nematode gene of the present invention include 5′ and 3′ terminal fusions as well as intra-sequence insertions of single or multiple nucleotides. Insertional nucleotide sequence variants are those in which one or more nucleotides are introduced into a predetermined site in the nucleotide sequence, although random insertion is also possible with suitable screening of the resulting product. Deletion variants are characterized by the removal of one or more nucleotides from the sequence. Nucleotide substitution variants are those in which at least one nucleotide in the sequence has been removed and a different nucleotide inserted in its place. Such a substitution may be silent (e.g., synonymous), meaning that the substitution does not alter the amino acid defined by the codon. Alternatively, substitutions are designed to alter one amino acid for another amino acid (e.g., non-synonymous). A non-synonymous substitution can be conservative or non-conservative. A substitution can be such that activity, e.g., a purine/pyrimidine phosphoribosyl transferase-like activity, is not impaired. A conservative amino acid substitution results in the alteration of an amino acid for a similar acting amino acid, or amino acid of like charge, polarity, or hydrophobicity, e.g., an amino acid substitution listed in Table 3 below. At some positions, even conservative amino acid substitutions can disrupt the activity of the polypeptide.

TABLE 3
Conservative Amino Acid Replacements

| | | |
|---|---|---|
| For Amino | Code | Replace with any of |

| | | |
|---|---|---|
| Alanine | Ala | Gly, Cys, Ser |
| Arginine | Arg | Lys, His |
| Asparagine | Asn | Asp, Glu, Gln, |
| Aspartic Acid | Asp | Asn, Glu, Gln |
| Cysteine | Cys | Met, Thr, Ser |
| Glutamine | Gln | Asn, Glu, Asp |
| Glutamic Acid | Glu | Asp, Asn, Gln |
| Glycine | Gly | Ala |
| Histidine | His | Lys, Arg |
| Isoleucine | Ile | Val, Leu, Met |
| Leucine | Leu | Val, Ile, Met |
| Lysine | Lys | Arg, His |
| Methionine | Met | Ile, Leu, Val |
| Phenylalanine | Phe | Tyr, His, Trp |
| Proline | Pro | |
| Serine | Ser | Thr, Cys, Ala |
| Threonine | Thr | Ser, Met, Val |
| Tryptophan | Trp | Phe, Tyr |
| Tyrosine | Tyr | Phe, His |
| Valine | Val | Leu, Ile, Met |

The current invention also embodies splice variants of nematode PPPT-like sequences.

Another aspect of the present invention embodies a polypeptide-encoding nucleic acid molecule that is capable of hybridizing under conditions of low stringency (or high stringency) to the nucleic acid molecule put forth in SEQ: ID NO: 1, 2, and/or 3, or their complements.

The nucleic acid molecules that encode for PPPT-like polypeptides may correspond to the naturally occurring nucleic acid molecules or may differ by one or more nucleotide substitutions, deletions, and/or additions. Thus, the present invention extends to genes and any functional mutants, derivatives, parts, fragments, homologs or analogs thereof or non-functional molecules. Such nucleic acid molecules can be used to detect polymorphisms of PPPT genes or PPPT-like genes, e.g., in other nematodes. As mentioned below, such molecules are useful as genetic probes; primer sequences in the enzymatic or chemical synthesis of the gene; or in the generation of immunologically interactive recombinant molecules. Using the information provided herein, such as the nucleotide sequence SEQ ID NO: 1, 2, and/or 3, a nucleic acid molecule encoding a PPPT-like molecule may be obtained using standard cloning and a screening techniques, such as a method described herein.

Nucleic acid molecules of the present invention can be in the form of RNA, such as mRNA, or in the form of DNA, including, for example, cDNA and genomic DNA obtained by cloning or produced synthetically. The DNA may be double-stranded or single-stranded. The nucleic acids may be in the form of RNA/DNA hybrids. Single-stranded DNA or RNA can be the coding strand, also referred to as the sense strand, or the non-coding strand, also known as the anti-sense strand.

One embodiment of the present invention includes a recombinant nucleic acid molecule, which includes at least one isolated nucleic acid molecule depicted in SEQ ID NO: 1, 2, and/or 3, inserted in a vector capable of delivering and maintaining the nucleic acid molecule into a cell. The DNA molecule may be inserted into an autonomously replicating factor (suitable vectors include, for example, pGEM3Z and pcDNA3, and derivatives thereof). The vector nucleic acid may be a bacteriophage DNA such as bacteriophage lambda or M13 and derivatives thereof. The vector may be either RNA or DNA, single- or double-stranded, either prokaryotic, eukaryotic, or viral. Vectors can include transposons, viral vectors, episomes, (e.g. plasmids), chromosomes inserts, and artificial chromosomes (e.g. BACs or YACs). Construction of a vector containing a nucleic acid described herein can be followed by transformation of a host cell such as a bacterium. Suitable bacterial hosts include, but are not limited to, E. coli. Suitable eukaryotic hosts include yeast such as S. cerevisiae, other fungi, vertebrate cells, invertebrate cells (e.g., insect cells), plant cells, human cells, human tissue cells, and whole eukaryotic organisms (e.g., a transgenic plant or a transgenic animal). Further, the vector nucleic acid can be used to generate a virus such as vaccinia or baculovirus.

The present invention also extends to genetic constructs designed for polypeptide expression. Generally, the genetic construct also includes, in addition to the encoding nucleic acid molecule, elements that allow expression, such as a promoter and regulatory sequences. The expression vectors may contain transcriptional control sequences that control transcriptional initiation, such as promoter, enhancer, operator, and repressor sequences. A variety of transcriptional control sequences are well known to those in the art and may be functional in, but are not limited to, a bacterium, yeast, plant, or animal cell. The expression vector can also include a translation regulatory sequence (e.g., an untranslated 5′ sequence, an untranslated 3′ sequence, a poly A addition site, or an internal ribosome entry site), a splicing sequence or splicing regulatory sequence, and a transcription termination sequence. The vector can be capable of autonomous replication or it can integrate into host DNA.

In an alternative embodiment, the DNA molecule is fused to a reporter gene such as β-glucuronidase gene, β-galactosidase (lacZ), chloramphenicol-acetyltransferase gene, a gene encoding green fluorescent protein (and variants thereof), or red fluorescent protein firefly luciferase gene, among others. The DNA molecule can also be fused to a nucleic acid encoding a polypeptide affinity tag, e.g. glutathione S-transferase (GST), maltose E binding protein, protein A, FLAG tag, hexa-histidine, or the influenza HA tag. The affinity tag or reporter fusion joins the reading frames of SEQ ID NO: 1, 2, and/or 3 to the reading frame of the reporter gene encoding the affinity tag such that a translational fusion is generated. Expression of the fusion gene results in translation of a single polypeptide that includes both a nematode PPPT-like region and reporter protein or affinity tag. The fusion can also join a fragment of the reading frame of SEQ ID NO: 1, 2, and/or 3. The fragment can encode a functional region of the PPPT-like polypeptides, a structurally-intact domain, or an epitope (e.g., a peptide of about 8, 10, 20, or 30 or more amino acids). A nematode PPPT-like nucleic acid that includes at least one of a regulatory region (e.g., a 5′ regulatory region, a promoter, an enhancer, a 5′ untranslated region, a translational start site, a 3′ untranslated region, a polyadenylation site, or a 3′ regulatory region) can also be fused to a heterologous nucleic acid. For example, the promoter of a PPPT-like nucleic acid can be fused to a heterologous nucleic acid, e.g., a nucleic acid encoding a reporter protein.

Suitable cells to transform include any cell that can be transformed with a nucleic acid molecule of the present invention. A transformed cell of the present invention is also herein referred to as a recombinant cell. Suitable cells can either be untransformed cells or cells that have already been transformed with at least one nucleic acid molecule. Suitable cells for transformation according to the present invention can either be: (i) endogenously capable of expressing the PPPT-like protein or; (ii) capable of producing such protein after transformation with at least one nucleic acid molecule of the present invention.

In an exemplary embodiment, a nucleic acid of the invention is used to generate a transgenic nematode strain, e.g., a transgenic C. elegans strain. To generate such a strain, nucleic acid is injected into the gonad of a nematode, thus generating a heritable extrachromosomal array containing the nucleic acid (see, e.g., Mello et al. (1991) EMBO J. 10:3959-3970). The transgenic nematode can be propagated to generate a strain harboring the transgene. Nematodes of the strain can be used in screens to identify inhibitors specific for a M. incognita, M. javanica, and/or H. glycines PPPT-like gene.

Oligonucleotides

Also provided are oligonucleotides that can form stable hybrids with a nucleic acid molecule of the present invention. The oligonucleotides can be about 10 to 200 nucleotides, about 15 to 120 nucleotides, or about 17 to 80 nucleotides in length, e.g., about 10, 20, 30, 40, 50, 60, 80, 100, 120 nucleotides in length. The oligonucleotides can be used as probes to identify nucleic acid molecules, primers to produce nucleic acid molecules, or therapeutic reagents to inhibit nematode PPPT-like protein activity or production (e.g., antisense, triplex formation, ribozyme, and/or RNA drug-based reagents). The present invention includes oligonucleotides of RNA (ssRNA and dsRNA), DNA, or derivatives of either. The invention extends to the use of such oligonucleotides to protect non-nematode organisms (for example, plants and animals) from disease, e.g., using a technology described herein. Appropriate oligonucleotide-containing therapeutic compositions can be administered to a non-nematode organism using techniques known to those skilled in the art, including, but not limited to, transgenic expression in plants or animals.

Primer sequences can be used to amplify a PPPT-like nucleic acid or fragment thereof. For example, at least 10 cycles of PCR amplification can be used to obtain such an amplified nucleic acid. Primers can be at least about 8-40, 10-30 or 14-25 nucleotides in length, and can anneal to a nucleic acid “template molecule”, e.g., a template molecule encoding a PPPT-like genetic sequence, or a functional part thereof, or its complementary sequence. The nucleic acid primer molecule can be any nucleotide sequence of at least 10 nucleotides in length derived from, or contained within sequences depicted in SEQ ID NO: 1, 2, and/or 3 and their complements. The nucleic acid template molecule may be in a recombinant form, in a virus particle, bacteriophage particle, yeast cell, animal cell, plant cell, fungal cell, or bacterial cell. A primer can be chemically synthesized by routine methods.

This invention embodies any PPPT-like sequences that are used to identify and isolate similar genes from other organisms, including nematodes, prokaryotic organisms, and other eukaryotic organisms, such as other animals and/or plants.

In another embodiment, the invention provides oligonucleotides that are specific for a M. incognita, M. javanica, and/or H. glycines PPPT-like nucleic acid molecule. Such oligonucleotides can be used in a PCR test to determine if a M. incognita, M. javanica, and/or H. glycines nucleic acid is present in a sample, e.g., to monitor a disease caused by M. incognita, M. javanica, and/or H. glycines.

Protein Production

Isolated PPPT-like proteins from nematodes can be produced in a number of ways, including production and recovery of the recombinant proteins and/or chemical synthesis of the protein. In one embodiment, an isolated nematode PPPT-like protein is produced by culturing a cell, e.g., a bacterial, fungal, plant, or animal cell, capable of expressing the protein, under conditions for effective production and recovery of the protein. The nucleic acid can be operably linked to a heterologous promoter, e.g., an inducible promoter or a constitutive promoter. Effective growth conditions are typically, but not necessarily, in liquid media comprising salts, water, carbon, nitrogen, phosphate sources, minerals, and other nutrients, but may be any solution in which PPPT-like proteins may be produced.

In one embodiment, recovery of the protein may refer to collecting the growth solution and need not involve additional steps of purification. Proteins of the present invention, however, can be purified using standard purification techniques, such as, but not limited to, affinity chromatography, thermaprecipitation, immunoaffinity chromatography, ammonium sulfate precipitation, ion exchange chromatography, filtration, electrophoresis, hydrophobic interaction chromatography, and others.

The PPPT-like polypeptide can be fused to an affinity tag, e.g., a purification handle (e.g., glutathione-S-reductase, hexa-histidine, maltose binding protein, dihydrofolate reductases, or chitin binding protein) or an epitope tag (e.g., c-myc epitope tag, FLAG™ tag, or influenza HA tag). Affinity tagged and epitope tagged proteins can be purified using routine art-known methods.

Antibodies Against PPPT-Like Polypeptides

Recombinant PPPT-like gene products or derivatives thereof can be used to produce immunologically interactive molecules, such as antibodies, or functional derivatives thereof. Useful antibodies include those that bind to a polypeptide that has substantially the same sequence as the amino acid sequences recited in SEQ ID NO: 4, 5, and/or 6, or that has at least 60% similarity over 50 or more amino acids to these sequences. In. a preferred embodiment, the antibody specifically binds to a polypeptide having the amino acid sequence recited in SEQ ID NO: 4, 5, and/or 6. The antibodies can be antibody fragments and genetically engineered antibodies, including single chain antibodies or chimeric antibodies that can bind to more than one epitope. Such antibodies may be polyclonal or monoclonal and may be selected from naturally occurring antibodies or may be specifically raised to a recombinant PPPT-like protein.

Antibodies can be derived by immunization with a recombinant or purified PPPT-like gene or gene product. As used herein, the term “antibody” refers to an immunoglobulin, or fragment thereof. Examples of antibody fragments include F(ab) and F(ab′)₂ fragments, particularly functional ones able to bind epitopes. Such fragments can be generated by proteolytic cleavage, e.g., with pepsin, or by genetic engineering. Antibodies can be polyclonal, monoclonal, or recombinant. In addition, antibodies can be modified to be chimeric, or humanized. Further, an antibody can be coupled to a label or a toxin.

Antibodies can be generated against a full-length PPPT-like protein, or a fragment thereof, e.g., an antigenic peptide. Such polypeptides can be coupled to an adjuvant to improve immunogenicity. Polyclonal serum is produced by injection of the antigen into a laboratory animal such as a rabbit and subsequent collection of sera. Alternatively, the antigen is used to immunize mice. Lymphocytic cells are obtained from the mice and fused with myelomas to form hybridomas producing antibodies.

Peptides for generating PPPT-like antibodies can be about 8, 10, 15, 20, 30 or more amino acid residues in length, e.g., a peptide of such length obtained from SEQ ID NO: 4, 5, and/or 6. Peptides or epitopes can also be selected from regions exposed on the surface of the protein, e.g., hydrophilic or amphipathic regions. An epitope in the vicinity of the active site can be selected such that an antibody binding such an epitope would block access to the active site. Antibodies reactive with, or specific for, any of these regions, or other regions or domains described herein are provided. An antibody to a PPPT-like protein can modulate a PPPT-like activity.

Monoclonal antibodies, which can be produced by routine methods, are obtained in abundance and in homogenous form from hybridomas formed from the fusion of immortal cell lines (e.g., myelomas) with lymphocytes immunized with PPPT-like polypeptides such as those set forth in SEQ ID NO: 4, 5, and/or 6.

In addition, antibodies can be engineered, e.g., to produce a single chain antibody (see, for example, Colcher et al. (1999) Ann N Y Acad Sci 880: 263-280; and Reiter (1996) Clin Cancer Res 2: 245-252). In still another implementation, antibodies are selected or. modified based on screening procedures, e.g., by screening antibodies or fragments thereof from a phage display library.

Antibodies of the present invention have a variety of important uses within the scope of this invention. For example, such antibodies can be used: (i) as therapeutic compounds to passively immunize an animal in order to protect the animal from nematodes susceptible to antibody treatment; (ii) as reagents in experimental assays to detect presence of nematodes; (iii) as tools to screen for expression of the gene product in nematodes, animals, fungi, bacteria, and plants; and/or (iv) as a purification tool of PPPT-like protein; (v) as PPPT inhibitors/activators that can be expressed or introduced into plants or animals for therapeutic purposes.

An antibody against a PPPT-like protein can be produced in a plant cell, e.g., in a transgenic plant or in culture (see, e.g., U.S. Pat. No. 6,080,560).

Antibodies that specifically recognize a M. incognita, M. javanica, and/or H. glycines PPPT-like proteins can be used to identify a M. incognita, M. javanica, and/or H. glycines nematodes, and, thus, can be used to monitor a disease caused by M. incognita, M. javanica, and/or H. glycines.

Nucleic Acids Agents

Also featured are isolated nucleic acids that are antisense to nucleic acids encoding nematode PPPT-like proteins. An Θantisense” nucleic acid includes a sequence that is complementary to the coding strand of a nucleic acid encoding a PPPT-like protein. The complementarity can be in a coding region of the coding strand or in a noncoding region, e.g., a 5′ or 3′ untranslated region, e.g., the translation start site. The antisense nucleic acid can be produced from a cellular promoter (e.g., a RNA polymerase II or III promoter), or can be introduced into a cell, e.g., using a liposome. For example, the antisense nucleic acid can be a synthetic oligonucleotide having a length of about 10, 15, 20, 30, 40, 50, 75, 90, 120 or more nucleotides in length.

An antisense nucleic acid can be synthesized chemically or produced using enzymatic reagents, e.g., a ligase. An antisense nucleic acid can also incorporate modified nucleotides, and artificial backbone structures, e.g., phosphorothioate derivative, and acridine substituted nucleotides.

Ribozymes. The antisense nucleic acid can be a ribozyme. The ribozyme can be designed to specifically cleave RNA, e.g., a PPPT-like mRNA. Methods for designing such ribozymes are described in U.S. Pat. No. 5,093,246 or Haselhoff and Gerlach (1988) Nature 334:585-591. For example, the ribozyme can be a derivative of Tetrahymena L-19 IVS RNA in which the nucleotide sequence of the active site is modified to be complementary to an PPPT-like nucleic acid (see, e.g., Cech et al. U.S. Pat. No. 4,987,071; and Cech et al. U.S. Pat. No. 5,116,742).

Peptide Nucleic acid (PNA). An antisense agent directed against a PPPT-like nucleic acid can be a peptide nucleic acid (PNA). See Hyrup et al. (1996) Bioorganic & Medicinal Chemistry 4: 5-23) for methods and a description of the replacement of the deoxyribose phosphate backbone for a pseudopeptide backbone. A PNA can specifically hybridize to DNA and RNA under conditions of low ionic strength as a result of its electrostatic properties. The synthesis of PNA oligomers can be performed using standard solid phase peptide synthesis protocols as described in Hyrup et al. (1996) supra; Perry-O'Keefe et al. Proc. Natl. Acad. Sci. 93: 14670-675.

RNA Mediated Interference (RNAi). A double stranded RNA (dsRNA) molecule can be used to inactivate a PPPT-like gene in a cell by a process known as RNA mediated-interference (RNAi; e.g., Fire et al. (1998) Nature 391:806-811, and Gonczy et al. (2000) Nature 408:331-336). The dsRNA molecule can have the nucleotide sequence of a PPPT-like nucleic acid described herein or a fragment thereof. The molecule can be injected into a cell, or a syncitia, e.g., a nematode gonad as described in Fire et al., supra.

Screening Assays

Another embodiment of the present invention is a method of identifying a compound capable of altering (e.g., inhibiting or enhancing) the activity of PPPT-like molecules. This method, also referred to as a “screening assay,” herein, includes, but is not limited to, the following procedure: (i) contacting an isolated PPPT-like protein with a test inhibitory compound, under conditions in which, in the absence of the test compound, the protein has PPPT-like activity; and (ii) determining if the test compound alters a PPPT-like activity. Suitable inhibitors or activators that alter a nematode PPPT-like activity include compounds that interact directly with a nematode PPPT-like protein, perhaps but not necessarily, in the active site. They can also interact with other regions of the nematode PPPT protein by binding to regions outside of the active site, for example, by allosteric interaction.

Compounds. A test compound can be a large or small molecule, for example, an organic compound with a molecular weight of about 100 to 10,000; 200 to 5,000; 200 to 2000; or 200 to 1,000 daltons. A test compound can be any chemical compound, for example, a small organic molecule, a carbohydrate, a lipid, an amino acid, a polypeptide, a nucleoside, a nucleic acid, or a peptide nucleic acid. Small molecules include, but are not limited to, metabolites, metabolic analogues, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds). A metabolite or metabolic analog can be a purine or pyrimidine (e.g., 8-azaadenine, 2,6-diaminopurine, 2-fluoroadenine), and derivatives thereof. Compounds and components for synthesis of compounds can be obtained from a commercial chemical supplier, e.g., Sigma-Aldrich Corp. (St. Louis, Mo.). The test compound or compounds can be naturally occurring, synthetic, or both. A test compound can be the only substance assayed by the method described herein. Alternatively, a collection of test compounds can be assayed either consecutively or concurrently by the methods described herein.

Examples of known inhibitors of PPPT proteins present in other organisms include [4-(3-nitroanilino)phthalic anhydride] (Somoza et al. (1998) Biochem. 37:5344-5348) and [(4′-phthalimido)carboxamido-3-(4-bromobenzyloxy)benzene] (Aronov et al. (2000) Biochem. 39:4684-4691). In addition, derivatives and mimetics of purines or pyrimidines can be screened and/or used.

A high-throughput method can be used to screen large libraries of chemicals. Such libraries of candidate compounds can be generated or purchased e.g., from Chembridge Corp. (San Diego, Calif.). Libraries can be designed to cover a diverse range of compounds. For example, a library can include 10,000, 50,000, or 100,000 or more unique compounds. Merely by way of illustration, a library can be constructed from heterocycles including pyridines, indoles, quinolines, furans, pyrimidines, triazines, pyrroles, imidazoles, naphthalenes, benzimidazoles, piperidines, pyrazoles, benzoxazoles, pyrrolidines, thiphenes, thiazoles, benzothiazoles, and morpholines. Alternatively, a class or category of compounds can be selected to mimic the chemical structures of purines or pyrmidines, [4-(3-nitroanilino)phthalic anhydride], [(4′-phthalimido)carboxamido-3-(4-bromobenzyloxy)benzene], or others. A library can be designed and synthesized to cover such classes of chemicals, e.g., as described in DeWitt et al. (1993) Proc. Natl. Acad. Sci. U.S.A. 90:6909-13; Erb et al. (1994) Proc. Natl. Acad. Sci. USA 91:11422-6; Zuckermann et al. (1994) J. Med. Chem. 37:2678-85; Cho et al. (1993) Science 261:1303-5; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233-51.

Organism-based Assays. Organisms can be grown in microtiter plates, e.g., 6-well, 32-well, 64-well, 96-well, 384-well plates.

In one embodiment, the organism is a nematode. The nematodes can be genetically modified. Non-limiting examples of such modified nematodes include: 1) nematodes or nematode cells (M. incognita, M. javanica, and/or H. glycines) having one or more PPPT-like genes inactivated (e.g., using RNA mediated interference); 2) nematodes or nematode cells expressing a heterologous PPPT-like gene, e.g., a PPPT-like gene from another species; and 3) nematodes or nematode cells having one or more endogenous PPPT-like genes inactivated and expressing a heterologous PPPT-like gene, e.g., a M. incognita, M. javanica, and/or H. glycines PPPT-like gene as described herein.

A plurality of candidate compounds, e.g., a combinatorial library, is screened. The library can be provided in a format that is amenable for robotic manipulation, e.g., in microtitre plates. Compounds can be added to the wells of the microtiter plates. Following compound addition and incubation, viability and/or reproductive properties of the nematodes or nematode cells are monitored.

The compounds can also be pooled, and the pools tested. Positive pools are split for subsequent analysis. Regardless of the method, compounds that decrease the viability or reproductive ability of nematodes, nematode cells, or progeny of the nematodes are considered lead compounds.

In another embodiment, the organism is a microorganism, e.g., a yeast or bacterium. For example, an E. coli strain having deletions or inactivating mutations in E. coli PPPT-like genes, but expressing a nematode PPPT-like gene can be used. The generation of such strains is routine in the art. As described above for nematodes and nematode cells, the microorganism can be grown in microtitre plates, each well having a different candidate compound or pool of candidate compounds. Growth is monitored during or after the assay to determine if the compound or pool of compounds is a modulator of a nematode PPPT-like polypeptide.

In Vitro Activity Assays. The screening assay can be an in vitro activity assay. For example, a nematode PPPT-like polypeptide can be purified as described above. The polypeptide can be disposed in an assay container, e.g., a well of a microtitre plate. A candidate compound can be added to the assay container, and the PPPT-like activity is measured. Optionally, the activity is compared to the activity measured in a control container in which no candidate compound is disposed or in which an inert or non-functional compound is disposed.

A PPPT-like activity assay can be carried by monitoring the pyrophosphorolysis of inosine monphosphate (IMP) or guanosine monophosphate (GMP). The formations of IMP and GMP can be followed spectrophotometrically at 245 and 257.5 nm, respectively (Hill (1970) Biochem. Pharmacol. 19: 545-557). Measurements can be carried out in 100 mM Tris-HCl, pH 7.4, and 12 mM MgCl₂ at 37° C. in a final volume of 1 ml.

The reverse reaction of IMP pyrophosphorolysis can be used to monitor PPPT-like polypeptide activity and can also be monitored spectrophotometrically. The production of hypoxanthine can be determined indirectly by the continuous spectrophotometric assay of uric acid formation in the presence of xanthine oxidase. The assay mixture can contain 100 mM Tris-HCl, pH 7.4, 12 mM MgCl₂, and 0.02 U/mL xanthine oxidase. The reaction can be initiated by the addition of purified phosphoribosyl transferase, and can be monitored at 293 nm at 37° C. GMP pyrophosphorolysis can be determined by continuous spectrophotometric assay of uric acid formulation in the presence of both guanase (0.01 U/ml) and xanthine oxidase (0.02 U/ml). Other conditions can be as described for the IMP pyrophosphorylsis assay (Yuan et al. (1992) Biochemistry 31:806-810).

In another embodiment, a purine phosphoribosyl transferase activity can be assayed in a mixture volume of 0.5 mL containing 0.05 μmole (1 μCi/umole) of ¹⁴C-labeled purine, 0.5 μmole of tetrasodium 5-phosphoribosyl-1-pyrophosphate, 0.1 M tris(hydroxymethyl) aminomethane-hydrochloride buffer (pH 8.0), 0.01 M magnesium sulfate, and 0.1 to 0.3 mg of protein of a cell free-extract (or an equivalent amount of pure protein). After cessation of the reaction, protein can be removed by centrifugation, and supernatent fluid can be applied to thin layer cellulose chromatogram sheet. The appropriate unlabeled purine ribonucleotide can be added at the point of each sample application and the sheets can be developed in 5% potassium phosphate-isoamyl alcohol. Nucleotides that are identified by UV absorption can be cut from the sheet, immersed in scintillation fluid, and counted (Gots et al. (1972) Journal of Bacteriology. 112:910-916). The kinetic and equilibrium parameters of the reaction can be determined, e.g., using art-known methods such as Lineweaver-Burk plots and Dixon plots. The assay can be used to measure inhibition coefficients, e.g., a Kᵢ, of a candidate compound, by measuring reaction rates at varying concentrations of the candidate compound.

This assay can be used to measure the ability of a candidate compound to inhibit the conversion of nucleosides to nucleotides by a nematode PPPT-like polypeptide.

In Vitro Binding Assays. The screening assay can also be a cell-free binding assay, e.g., an assay to identify compounds that bind a nematode PPPT-like polypeptide. For example, a nematode PPPT-like polypeptide can be purified and labeled. The labeled polypeptide is contacted to beads; each bead has a tag detectable by mass spectroscopy, and a test compound, e.g., a compound synthesized by combinatorial chemical methods. Beads to which the labeled polypeptide is bound are identified and analyzed by mass spectroscopy. The beads can be generated using “split-and-pool” synthesis. The method can further include a second assay (e.g., the PPPT activity assay described above) to determine if the compound alters the activity of the PPPT-like polypeptide.

Optimization of a Compound. Once a lead compound has been identified, standard principles of medicinal chemistry can be used to produce derivatives of the compound. Derivatives can be screened for improved pharmacological properties, for example, efficacy, pharmacokinetics, stability, solubility, and clearance. The moieties responsible for a compound's activity in the above-described assays can be delineated by examination of structure-activity relationships (SAR) as is commonly practiced in the art. One can modify moieties on a lead compound and measure the effects of the modification on the efficacy of the compound to thereby produce derivatives with increased potency. For an example, see Nagarajan et al. (1988) J. Antibiot. 41:1430-1438. A modification can include N-acylation, amination, amidation, oxidation, reduction, alkylation, esterification, and hydroxylation. Furthermore, if the biochemical target of the lead compound is known or determined, the structure of the target and the lead compound can inform the design and optimization of derivatives. Molecular modeling software is commercially available (e.g., Molecular Simulations, Inc.). “SAR by NMR”, as described in Shuker et al. (1996) Science 274:1531-1534, can be used to design ligands with increased affinity, by joining lower-affinity ligands.

A preferred compound is one that inhibits a PPPT-like polypeptide and that is not substantially toxic to plants, animals, or humans. By “not substantially toxic” it is meant that the compound does not substantially affect the respective plant, animal, or human PPPT proteins. Thus, particularly desirable inhibitors of M. incognita, M. javanica, and/or H. glycines PPPT do not substantially inhibit PPPT-like polypeptides of cotton, tobacco, pepper, tomato, and/or soybean, for example.

Standard pharmaceutical procedures can be used to assess the toxicity and therapeutic efficacy of a modulator of a PPPT-like activity. The LD50 (the dose lethal to 50% of the population) and the ED50 (the dose therapeutically effective in 50% of the population can be measured in cell cultures, experimental plants (e.g., in laboratory or field studies), or experimental animals. Optionally, a therapeutic index can be determined which is expressed as the ratio: LD50/ED50. High therapeutic indices are indicative of a compound being an effective PPPT-like inhibitor, while not causing undue toxicity or side-effects to a subject (e.g., a host plant or host animal).

Alternatively, the ability of a candidate compound to modulate a non-nematode PPPT-like polypeptide is assayed, e.g., by a method described herein. For example, the inhibition constant of a candidate compound for a mammalian PPPT-like polypeptide or a plant PPPT-like polypeptide (e.g., a PPPT-like polypeptide from cotton, tobacco, pepper, tomato; purine/pyrimidine phosphoribosyl transferase (Soybean P52418 GI: 1709918, Tobacco P93394 GI: 6647900) can be measured and compared to the inhibition constant for a nematode PPPT-like polypeptide. (An Advanced Treatise on Meloidogyne, Vol. 1, Sasser and Carter, North Carolina State University Graphics, 1985; Root-Knot Nematodes: A global menace to crop production. Sasser. Plant Disease 64:36-41, 1980.)

The aforementioned analyses can be used to identify and/or design a modulator with specificity for nematode PPPT-like polypeptide over plant or other animal (e.g., mammalian) PPPT-like polypeptides. Suitable nematodes to target are any nematodes with the PPPT-like proteins or proteins that can be targeted by a compound that otherwise inhibits, reduces, activates, or generally effects the activity of nematode PPPT proteins.

Inhibitors of nematode PPPT-like proteins can also be used to identify PPPT-like proteins in the nematode or other organisms using procedures known in the art, such as affinity chromatography. For example, a known inhibitor may be linked to a resin and a nematode extract passed over the resin, allowing any PPPT-like proteins that bind the inhibitor to bind the resin. Subsequent biochemical techniques familiar to those skilled in the art can be performed to purify and identify bound PPPT-like proteins.

Agricultural Compositions

A compound that is identified as a PPPT-like polypeptide inhibitor can be formulated as a composition that is applied to plants, soil, or seeds in order to confer nematode resistance. The composition can be prepared in a solution, e.g., an aqueous solution, at a concentration from about 0.005% to 10%, or about 0.01% to 1%, or about 0.1% to 0.5% by weight. The solution can include an organic solvent, e.g., glycerol or ethanol. The composition can be formulated with one or more agriculturally acceptable carriers. Agricultural carriers can include: clay, talc, bentonite, diatomaceous earth, kaolin, silica, benzene, xylene, toluene, kerosene, N-methylpyrrolidone, alcohols (methanol, ethanol, isopropanol, n-butanol, ethylene glycol, propylene glycol, and the like), and ketones (acetone, methylethyl ketone, cyclohexanone, and the like). The formulation can optionally further include stabilizers, spreading agents, wetting extenders, dispersing agents, sticking agents, disintegrators, and other additives, and can be prepared as a liquid, a water-soluble solid (e.g., tablet, powder or granule), or a paste.

Prior to application, the solution can be combined with another desired composition such as another antihelmintic agent, germicide, fertilizer, plant growth regulator and/or the like. The solution may be applied to the plant tissue, for example, by spraying, e.g., with an atomizer, by drenching, by pasting, or by manual application, e.g., with a sponge. The solution can also be distributed from an airborne source, e.g., an aircraft or other aerial object, e.g., a fixture mounted with an apparatus for spraying the solution, the fixture being of sufficient height to distribute the solution to the desired plant tissues. Alternatively, the composition can be applied to plant tissue from a volatile or airborne source. The source is placed in the vicinity of the plant tissue and the composition is dispersed by diffusion through the atmosphere. The source and the plant tissue to be contacted can be enclosed in an incubator, growth chamber, or greenhouse, or can be in sufficient proximity that they can be outdoors.

If the composition is distributed systemically thorough the plant, the composition can be applied to tissues other than the leaves, e.g., to the stems or roots. Thus, the composition can be distributed by irrigation. The composition can also be injected directly into roots or stems.

A skilled artisan would be able to determine an appropriate dosage for formulation of the active ingredient of the composition. For example, the ED50 can be determined as described above from experimental data. The data can be obtained by experimentally varying the dose of the active ingredient to identify a dosage effective for killing a nematode, while not causing toxicity in the host plant or host animal (i.e. non-nematode animal).

A number of embodiments of the invention have been described. Nevertheless, it will be understood that various modifications may be made without departing from the spirit and scope of the invention. Accordingly, other embodiments are within the scope of the following claims.

1-13. (canceled)

14. A method comprising:
(a) providing a polypeptide comprising the amino acid sequence of SEQ ID NO:4;
(b) contacting a test compound to the polypeptide;
(c) measuring the binding of the test compound to the polypeptide; and
(d) measuring the phosphoribosyl transferase activity of the polypeptide in the presence of the test compound.

15. The method of claim 14, further comprising:
(e) measuring the phosphoribosyl transferase activity of the polypeptide in the absence of the test compound.

16. The method of claim 14, further comprising:
(e) contacting the test compound to a second polypeptide selected from the group consisting of a plant phosphoribosyl transferase and a mammalian phosphoribosyl transferase; and
(f) measuring the phosphoribosyl transferase activity of the second polypeptide in the presence of the test compound.

17. A method comprising:
(a) providing a polypeptide comprising the amino acid sequence of SEQ ID NO:4;
(b) contacting a test compound to the polypeptide; and
(c) measuring the phosphoribosyl transferase activity of the polypeptide, wherein a change in phosphoribosyl transferase activity relative to the phosphoribosyl transferase activity of the polypeptide in the absence of the test compound indicates that the test compound alters the activity of the polypeptide.

18. A method comprising:
(a) providing a polypeptide comprising the amino acid sequence of SEQ ID NO:4;
(b) contacting a test compound to the polypeptide;
(c) measuring the phosphoribosyl transferase activity of the polypeptide in the presence of the test compound;
(d) identifying a test compound as a inhibitor of the polypeptide when phosphoribosyl transferase activity of the peptide in the presence of the compound is less than the phosphoribosyl transferase activity of the polypeptide in the absence of the test compound; and
(e) contacting a candidate inhibitor of the polypeptide identified in (d) with a second polypeptide selected from the group consisting of a plant PPPT polypeptide and a mammalian PPPT polypeptide; and
(f) measuring the phosphoribosyl transferase activity of the second polypeptide in the presence of the test compound.
